(19) United States
(12) Patent Application Publication
(10) Pub. No.: US 2005/0260132 A1
(43) Pub. Date: Nov. 24, 2005

(54) Monoclonal antibodies directed to receptor protein tyrosine phosphatase zeta

(76) Inventors: Daniel Chin, Foster City, CA (US); Erik Foehr, Novato, CA (US); Sabine Mueller, San Francisco, CA (US); Thorsten Melcher, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/097,728

(22) Filed: Mar. 31, 2005

Related U.S. Application Data
(63) Continuation-in-part of application No. 10/652,981, filed on Aug. 28, 2003.
Application No. 10/652,981 is a continuation of application No. 09/983,000, filed on Oct. 17, 2001, now abandoned.

(51) Int. Cl. A61K 51/00; A61K 39/395

(52) U.S. Cl. 424/1.49; 424/146.1

(57) ABSTRACT

The present invention relates to a method of inhibiting growth of tumor cells which overexpress a receptor protein tyrosine phosphatase zeta (PTPζ) by treatment of the cells with antibodies which recognize PTPζ and/or inhibit PTPζ function. The present invention also provides compounds and pharmaceutically acceptable compositions for administration in the methods of the invention. The present invention also provides novel splice variants of protein PTPζ, PTPζ SM1 and PTPζ SM2. Nucleic acid probes specific for the spliced mRNA encoding these variants and affinity reagents specific for the novel proteins are also provided.

### FIELD OF USE

The present invention relates to a method of inhibiting growth of tumor cells that overexpress a receptor protein tyrosine phosphatase zeta (PTPζ), by treatment of the cells with antibodies that recognize PTPζ and/or inhibit PTPζ function. Specifically, the present invention relates to the use of immunotherapeutic and immunoimaging agents that specifically bind receptor protein tyrosine phosphatase zeta (PTPζ) for the treatment and visualization of brain tumors in patients. The present invention also provides compounds and pharmaceutically acceptable compositions for administration.

### BACKGROUND OF THE INVENTION

Brain Tumor Biology and Etiology

Brain tumors are considered to have one of the least favorable prognoses for long term survival: the average life expectancy of an individual diagnosed with a central nervous system (CNS) tumor is just eight to twelve months. Several unique characteristics of both the brain and its particular types of neoplastic cells create daunting challenges for the complete treatment and management of brain tumors. Among these are 1) the physical characteristics of the intracranial space, 2) the relative biological isolation of the brain from the rest of the body, 3) the relatively essential and irreplaceable nature of the organ mass, and 4) the unique nature of brain tumor cells.

First and foremost, the intracranial space and physical layout of the brain create significant obstacles to treatment and recovery. The brain is made of, primarily, astrocytes (which make up the majority of the brain mass, and serve as a scaffold and support for the neurons), neurons (which carry the actual electrical impulses of the nervous system), and a minor contingent of other cells such as insulating oligodendrocytes (which produce myelin). These cell types give rise to primary brain tumors (e.g., astrocytomas, neuroblastomas, glioblastomas, oligodendrogliomas, etc.) Although the World Health Organization has recently established standard guidelines, the nomenclature for brain tumors is somewhat imprecise, and the terms astrocytoma and glioblastoma are often used broadly. The brain is encased in the relatively rigid shell of the skull, and is cushioned by the cerebrospinal fluid, much like a fetus in the womb. Because of the relatively small volume of the skull cavity, minor changes in the volume of tissue in the brain can dramatically increase intracranial pressure, causing damage to the entire organ (i.e., “water on the brain”). Thus, even small tumors can have a profound and adverse affect on the brain's function. In contrast, tumors in the relatively distensible abdomen may reach several pounds in size before the patient experiences adverse symptoms. The cramped physical location of the cranium also makes surgery and treatment of the brain a difficult and delicate procedure. However, because of the dangers of increased intracranial pressure from the tumor, surgery is often the first strategy of attack in treating brain tumors.

In addition to its physical isolation, the brain is chemically and biologically isolated from the rest of the body by the so-called “Blood-Brain-Barrier” (or BBB). This physiological phenomenon arises because of the “tightness” of the epithelial cell junctions in the lining of the blood vessels in the brain. Although nutrients, which are actively transported across the cell lining, may reach the brain, other molecules from the bloodstream are excluded. This prevents toxins, viruses, and other potentially dangerous molecules from entering the brain cavity. However, it also prevents therapeutic molecules, including many chemotherapeutic agents that are useful in other types of tumors, from crossing into the brain. Thus, many therapies directed at the brain must be delivered directly into the brain cavity (e.g., by an Ommaya reservoir), or administered in elevated dosages to ensure the diffusion of an effective amount across the BBB.

With the difficulties of administering chemotherapies to the brain, radiotherapy approaches have also been attempted. However, the amount of radiation necessary to completely destroy potential tumor-producing cells also produce unacceptable losses of healthy brain tissue. The retention of patient cognitive function while eliminating the tumor mass is another challenge to brain tumor treatment. Neoplastic brain cells are often pervasive, and travel throughout the entire brain mass. Thus, it is impossible to define a true “tumor margin,” unlike, for example, in lung or bladder cancers. Unlike reproductive (ovarian, uterine, testicular, prostate, etc.), breast, kidney, or lung cancers, the entire organ, or even significant portions, cannot be removed to prevent the growth of new tumors. In addition, brain tumors are very heterogeneous, with different cell doubling times, treatment resistances, and other biochemical idiosyncrasies between the various cell populations that make up the tumor. This pervasive and variable nature greatly adds to the difficulty of treating brain tumors while preserving the health and function of normal brain tissue.

Although current surgical methods offer considerably better post-operative life for patients, the current combination therapy methods (surgery, low-dosage radiation, and chemotherapy) have only improved the life expectancy of patients by one month, as compared to the methods of 30 years ago. Without effective agents to prevent the growth of brain tumor cells that are present outside the main tumor mass, the prognosis for these patients cannot be significantly improved. Although some immuno-affinity agents have been proposed and tested for the treatment of brain tumors, see, e.g., the tenascin-targeting agents described in U.S. Pat. No. 5,624,659, these agents have not proven sufficient for the treatment of brain tumors. Thus, therapeutic agents which are directed towards new molecular targets, and are capable of specifically targeting and killing brain tumor cells, are urgently needed for the treatment of brain tumors.

Protein Tyrosine Phosphatase Receptor Zeta (PTPζ)

Vital cellular functions, such as cell proliferation and signal transduction, are regulated in part by the balance between the activities of protein kinases and protein phosphatases. These protein-modifying enzymes add or remove a phosphate group from serine, threonine, or tyrosine residues in specific proteins. Some tyrosine kinases (PTK's) and phosphatases (PTPase's) have been theorized to have a role in some types of oncogenesis, which is thought to result from an imbalance in their activities. There are two classes of PTPase molecules: low molecular weight proteins with a single conserved phosphatase domain such as T-cell protein-tyrosine phosphatase (PTPT; MIM 176887), and high molecular weight receptor-linked PTPases with two tandemly repeated and conserved phosphatase domains separated by 56 to 57 amino acids. Examples of this latter group of receptor proteins include: leukocyte-common antigen (PTPRC; MIM 151460) and leukocyte antigen related tyrosine phosphatase (PTPRF; MIM 179590).

Protein tyrosine phosphatase zeta (PTPζ), also known as PTPRZ, HPTP-ZETA, HPTPZ, RPTP-BETA(β), or RPTPB, was isolated as a cDNA sequence by two groups in the early nineties. The complete cDNA sequence of the protein is provided in SEQ ID NO. 1, and the complete deduced amino acid sequence is provided in SEQ ID NO. 2. Splicing variants and features are indicated in the sequences. Levy et al. (“The cloning of a receptor-type protein tyrosine phosphatase expressed in the central nervous system” J. Biol. Chem. 268: 10573-10581, (1993)) isolated cDNA clones from a human infant brain mRNA expression library, and deduced the complete amino acid sequence of a large receptor-type protein tyrosine phosphatase containing 2,307 amino acids.

Levy found that the protein, which they designated RPTP-β (PTPζ), is a transmembrane protein with 2 cytoplasmic PTPase domains and a 1,616-amino acid extracellular domain. As in PTP-γ (MIM 176886), the 266 N-terminal residues of the extracellular domain have a high degree of similarity to carbonic anhydrases (see MIM 114880). The human gene encoding PTPζ has been mapped to chromosome 7q31.3-q32 by chromosomal in situ hybridization (Ariyama et al., “Assignment of the human protein tyrosine phosphatase, receptor-type, zeta (PTPRZ) gene to chromosome band 7q31.3” Cytogenet. Cell Genet. 70: 52-54 (1995)). Northern blot analysis has shown that PTPζ is expressed only in the human central nervous system. By in situ hybridization, Levy et al. (1993) localized the expression to different regions of the adult mouse brain, including the Purkinje cell layer of the cerebellum, the dentate gyrus, and the subependymal layer of the anterior horn of the lateral ventricle. Levy stated that this was the first mammalian tyrosine phosphatase whose expression is restricted to the nervous system. In addition, high levels of expression in the murine embryonic brain suggest an important role in CNS development.

Northern analysis has shown three splice variants: the extracellular proteoglycan phosphacan, which contains the full extracellular region of the protein, and the long (α) and short (β) forms of the transmembrane phosphatase. The β form lacks the extracellular 860 aa long insert domain of the protein, therefore lacks several glycosylation sites. PCR studies of the gene in rat genomic DNA indicated that there are no introns at the putative 5′ and 3′ splice sites or in the 2.6 kb segment which is deleted in the short transmembrane protein. The phosphatases and the extracellular proteoglycan have different 3′-untranslated regions. Additional alternative mRNA splicing is likely to result in the deletion of a 7 amino acid insert from the intracellular juxtamembrane region of both long and short phosphatase isoforms. Simultaneous quantitation of the three major isoforms indicated that the mRNA encoding phosphacan had the highest relative abundance in the CNS while that encoding the short phosphatase isoform was most abundant relative to the other PTPζ variants in the CNS.

The sequences of these polynucleotides, and the encoded polypeptides, are provided as SEQ ID NO:1; SEQ ID NO:3 and SEQ ID NO:5 for the nucleotides sequences, and SEQ ID NO:2, 4 and 6 for the respective encoded products.

The transmembrane forms of PTPζ are expressed on the migrating neurons especially at the lamellipodia along the leading processes. PTPζ is postulated to be involved in the neuronal migration as a neuronal receptor of pleiotrophin distributed along radial glial fibers. PTPζ has been shown to be highly expressed in radial glia and other forms of glial cells that play an important role during development. The anti-PTPζ staining localizes to the radial processes of these cells, which act as guides during neuronal migration and axonal elongation. The pattern of PTPζ expression has also been shown to change with the progression of glial cell differentiation.

The three splice variants of PTPζ have been shown to have different spatial and temporal patterns of expression in the developing brain. The 9.5-kb and 6.4-kb transcripts, which encode the α and β transmembrane protein tyrosine phosphatases, were predominantly expressed in glial progenitors located in the subventricular zone. The 8.4-kb transcript, which encodes the secreted chondroitin sulfate proteoglycan phosphacan, was expressed at high levels by more mature glia that have migrated out of the subventricular zone. The three transcripts have also been shown to be differentially expressed in glial cell cultures.

In knockout studies, PTPζ-deficient mice were viable, fertile, and showed no gross anatomical alterations in the nervous system or other organs. Therefore, it was deduced that PTPζ is not essential for neurite outgrowth and node formation in mice. The ultrastructure of nerves of the central nervous system in PTPζ-deficient mice suggests a fragility of myelin. However, conduction velocity was not altered. The normal development of neurons and glia in PTPζ deficient mice was thought to indicate that PTPζ function is not necessary for these processes in vivo, or that a loss of PTPζ can be compensated for by other protein tyrosine phosphatases expressed in the nervous system.

Following CNS injury, robust induction of phosphatase forms of PTPζ mRNA has been observed in areas of axonal sprouting, and of both phosphatases and phosphacan mRNAs in areas of glial scarring. This is thought to imply that the encoded proteins and the cell adhesion molecules and extracellular matrix proteins to which they bind may contribute to recovery from injury and perhaps also to the regulation of axonal regrowth in the nervous system. Following peripheral nerve crush, all PTPζ mRNAs, including phosphacan and the phosphatase variants with and without the 21 base insert, were observed to be significantly induced in the distal segments of the sciatic nerve with a time course that correlated well with the response of Schwann cells to this injury.

The extracellular domains of PTPζ have been shown to be capable of binding to several cell adhesion molecules. Phosphacan, which is the shortest, secreted form of PTPζ, containing the full extracellular region, previously was designated 3F8 and 6B4 chondroitin sulfate proteoglycan or 3H1 keratin sulfate proteoglycan depending on the glycosylation status. It is synthesized mainly by glia and binds to neurons and to the neural cell adhesion molecules Ng-CAM/L1, NCAM, TAG-1/axonin-1, to tenascin-C and R, to amphoterin and pleiotrophin/heparin-binding growth-associated molecule (HB-GAM) (amphoterin and pleiotrophin are heparin-binding proteins that are developmentally regulated in brain and functionally involved in neurite outgrowth). Binding of phosphacan to Ng-CAM/L1, NCAM, and tenascin-C (FNIII domain) is mediated by complex-type N-linked oligosaccharides on the proteoglycan. Phosphacan, shows saturable, reversible, high-affinity binding to fibroblast growth factor-2 (FGF-2). The interaction is mediated primarily through the core protein. Immunocytochemical studies have also shown an overlapping localization of FGF-2 and phosphacan in the developing central nervous system. The core protein of phosphacan may also regulate the access of FGF-2 to cell surface signaling receptors in nervous tissue.

The carbonic anhydrase (CAH) domain of PTPζ has been shown to bind specifically to contactin. Contactin is a 140 kDa GPI membrane-anchored neuronal cell recognition protein expressed on the surface of neuronal cells. The CAH domain of RPTP zeta was shown to induce cell adhesion and neurite growth of primary tectal neurons, and differentiation of neuroblastoma cells. These responses were blocked by antibodies against contactin, demonstrating that contactin is a neuronal receptor for RPTP zeta. Caspr ((p190/Caspr, a contactin-associated transmembrane receptor) and contactin exist as a complex in rat brain and are bound to each other by means of lateral (cis) interactions in the plasma membrane. The extracellular domain of Caspr contains a neurophilin/coagulation factor homology domain, a region related to fibrinogen beta/gamma, epidermal growth factor-like repeats, neurexin motifs as well as unique PGY repeats found in a molluscan adhesive protein. The cytoplasmic domain of Caspr contains a proline-rich sequence capable of binding to a subclass of SH3 domains of signaling molecules. Caspr may function as a signaling component of contactin, enabling recruitment and activation of intracellular signaling pathways in neurons. The role of the extracellular domains in neural adhesion and neurite growth induction was investigated by the use of fusion protein constructs. The results suggested that binding of glial PTPζ to the contactin/Nr-CAM complex is important for neurite growth and neuronal differentiation.

PTPζ was shown to bind to a heparin-binding growth factor midkine through the chondroitin sulfate portion of the receptor. The interactions of pleiotrophin (PTN) with the receptor in U373-MG cells was also studied. Pleiotrophin was shown to bind to the spacer domain. Results suggested that PTN signals through “ligand-dependent receptor inactivation” of PTPζ and disrupts its normal roles in the regulation of steady-state tyrosine phosphorylation of downstream signaling molecules. PTN was shown to bind to and functionally inactivate the catalytic activity of PTPζ. An active site-containing domain of PTPζ both binds β-catenin and functionally reduces its levels of tyrosine phosphorylation when added to lysates of pervanadate-treated cells. In unstimulated cells, PTPζ was shown to be intrinsically active, and thought to function as an important regulator in the reciprocal control of the steady-state tyrosine phosphorylation levels of β-catenin by tyrosine kinases and phosphatases.

Using the yeast substrate-trapping system, several substrate candidates for PTPζ were isolated. The results indicated that GIT1/Cat-1 is a substrate molecule of PTPζ. In addition, PTPζ was shown to bind to the PSD-95/SAP90 family through the second phosphatase domain. Immunohistochemical analysis revealed that PTPζ and PSD-95/SAP90 are similarly distributed in the dendrites of pyramidal neurons of the hippocampus and neocortex. Subcellular fractionation experiments indicated that PTPζ is concentrated in the postsynaptic density fraction. These results suggested that PTPζ is involved in the regulation of synaptic function as postsynaptic macromolecular complexes with PSD-95/SAP90.

Voltage-gated sodium channels in brain neurons were also found to associate with the membrane bound forms of PTPζ and phosphacan. Both the extracellular domain and the intracellular catalytic domain of PTPζ interacted with sodium channels. Sodium channels were tyrosine phosphorylated and were modulated by the associated catalytic domains of PTPζ.

### SUMMARY OF THE INVENTION

The present invention provides novel methods and reagents for specifically targeting tumor cells for both therapeutic and imaging purposes, using antibodies specific for the receptor protein tyrosine phosphatase zeta (PTPζ), including the two novel isoforms PTPζ SM1 and SM2. These targets have been identified by the applicants as being overexpressed in brain and other tumors, and thus allow for the selective inhibition of cell function or selective marking for visualization with therapeutic or visualizing compositions which have a specific affinity for these protein targets.

In one embodiment of the invention, the therapeutic agent comprises an antibody specific for the ectodomain of the PTPζ short form (also referred to as the PTPζ-β form). This domain includes residues 26-774 of SEQ ID NO:2. Preferred antibodies bind to conformational epitopes present on the membrane-bound PTPζ protein, as it is presented by live tumor cells. Other useful attributes of the antibodies of the invention include high binding affinity, e.g. of at least about 10 nM K_{D}; and internalize upon binding to live cells. Such antibodies may be used in an unmodified form, or conjugated to various cytotoxic or imaging moieties.

Antibodies specific for PTPζ are useful in the treatment of tumors in patients. In one embodiment, the tumor is a brain tumor, including astrocytomas such as grade II astrocytoma, grade III anaplastic astrocytoma; and grade IV glioblastoma multiforme (GBM). In other embodiments of the invention, the tumor is a carcinoma, which tumors include invasive ductal carcinoma of the breast; colon adenocarcinoma; transitional carcinoma of the bladder; and squamous cell carcinoma of the oral cavity and pharanx. The methods comprise administering an effective amount of a composition, comprising an antibody specific for PTPζ, which antibody is optionally conjugated to a cytotoxic moiety, and a pharmaceutically acceptable carrier, to a patient in need thereof. Administration of the therapeutic composition may be by any acceptable means. One preferred method for administration is by intrathecal administration, although intratumor, or intravascular administration also find use.

The antibodies of the invention find use in the visualization of tumors. These methods generally comprise administering an effective amount of an imaging compound of the general formula α(PTPζ)I, where I is an imaging moiety, in a pharmaceutically acceptable carrier to the patient, and then visualizing the imaging moieties of the compound. Administration of the imaging composition may be by any acceptable means. Intravascular administration of the imaging composition is preferred in these methods, although intrathecal administration is also preferred. Preferred methods of visualizing the imaging moieties of the compounds include radiographic imaging techniques (e.g., x-ray imaging and scintillation imaging techniques), positron-emission tomography, magnetic resonance imaging techniques, and direct or indirect, e.g., endoscopic, visual inspection.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIG. 1: A diagram of the three known splicing variant isoforms of PTPζ. The approximate position of the domains of the isoforms is indicated underneath the isoforms, as well as the approximate exon size (for size reference, exon 12 is 3.6 kilobases.) Isoform PTPζ-α is the full length isoform, which contains the primary amino acid sequence aa 25-2314 of SEQ ID NO. 2 (aa 1-24 are a signal polypeptide). In Isoform PTPζ-β, aa 755-1614 are missing. Isoform PTPζ-S (phosphacan), is a secreted isoform which comprise the extracellular domains of PTPζ-α, in which the transmembrane and cytosol domains are missing.

FIG. 2: A diagram of the two newly discovered splicing variant isoforms of PTPζ. The approximate position of the domains of the isoforms is indicated underneath the isoforms, as well as the approximate exon size (for size reference, exon 12 is 3.6 kilobases.) SM 1 fails to splice correctly after the 9^{th} exon, yielding an mRNA with two extra codons followed by a stop codon after the normal terminus of exon 9. SM 2 contains a 116 nucleotide insertion from between exons 23 & 24.

FIG. 3: A diagram comparing the three known PTPζ isoforms with the two novel isoforms.

FIG. 4: A subset of PTPζ antibodies generated from the immunization of mice with PTPζ- β and were tested by ELISA for selectivity to either recombinant PTPζ-β (black bars) or a non-specific control protein (grey bars). An IgG1 negative control isotype was included as a reference (error bars±S.D.)

FIG. 5: Flow cytometry analysis of PTPζ antibody binding to live U87 glioma cells. Human U87 glioma cells were stained with control IgG1, 1B9G4, 7A9B5, or 7E4B11 as indicated and detected with a fluorescent secondary antibody. Live cells were gated from dead cells and the fluorescent intensity of the cell population measured using flow cytometry.

FIG. 6A-6B: PTPζ antibody mediated tumor cell killing. (A) For indirect immunotoxin experiments, U87 glioma cells are treated with PTPζ antibodies as well as positive control antibodies, anti-EGFR and anti-CD71. The negative controls included media alone and an isotype control IgG1. Following primary Ab treatment, the cells are subsequently incubated with media (black bars) or a saporin toxin conjugated anti-mouse antibody (grey bars), (error bars±S.D.). (B) For direct immunotoxin experiments, purified 7E4B11 and 7A9B5 antibodies were directly conjugated to Saporin and evaluated in cell culture for the ability to kill glioma cells. In this experiment glioma cells are treated with 7E4B11 and 7A9B5 immunotoxins along with control immunotoxins, non-specific IgG-SAP (Neg. Ctrl), DAT-SAP (Pos. Ctrl) as well as vehicle (error bars±S.D.).

FIG. 7: Effect of 7E4B11 on tumor growth in soft agar. U87 cells suspended in soft agar were treated with media containing IgG1 (20 82 g/ml), EGFR-528 (20 μg/ml), or 7E4B11 (20 μg/ml). The cells were incubated for 21 days with media treatment changes every 3-4 days. At that time colonies were stained and imaged using light microscopy as shown here.

FIG. 8A-8B: Effect of 7E4B11-SAP on tumor growth delay in established U87 xenografts. (A and B) Mice received intratumoral injections twice a week for two weeks once tumors had reached a mean tumor volume of 130 mm3. The immunotoxins 7E4B11-SAP (30 μg/dose), and IgG-SAP (30 μg/dose) were tested alongside the vehicle control (PBS) for tumor growth delay of established tumors. (A) Data are expressed as mean tumor volume versus time and (B) as time to end point (growth of tumor to 1500 mm3).

### DETAILED DESCRIPTION OF THE INVENTION

Applicants have identified the receptor protein tyrosine phosphatase zeta (PTPζ), including the two novel isoforms PTPζ SM1 and SM2 as being differentially regulated between cancer tissue and brain tissue. Cancers shown to have differential expression include astrocytomas, and carcinomas, including carcinoma, which tumors include invasive ductal carcinoma of the breast; colon adenocarcinoma; transitional carcinoma of the bladder; and squamous cell carcinoma of the oral cavity and pharanx.

In one embodiment of the invention, an antibody is provided that is specific for the ectodomain of the PTPζ short form, which domain includes residues 26-774 of SEQ ID NO:2. The antibody preferably binds to conformational epitopes present on the PTPζ protein as it is presented by live tumor cells. Other useful attributes of the antibodies of the invention include high binding affinity, e.g. of at least about 10 nM K_{D}; and internalization upon binding to live cells. Such antibodies specific for PTPζ are useful in the treatment of tumors in patients. The methods comprise administering an effective amount of a composition, comprising an antibody specific for PTPζ, which antibody is optionally conjugated to a cytotoxic moiety, and a pharmaceutically acceptable carrier, to a patient in need thereof.

Applicants have performed differential cloning between cancerous and normal brains and have identified the PTPζ genes by DNA sequence analysis. The overexpressed PTPζ genes and protein products mediate the initiation and progression of tumors. PTPζ on the cell surface provides excellent targets for immunotherapeutic agents that either deliver cytotoxic agents to directly promote tumor cell death, or that alter PTPζ function to inhibit the normal physiology of the tumor cell. In addition, immunoimaging agents targeted to PTPζ may be utilized to visualize the tumor mass either in diagnostic methods (e.g., magnetic resonance imaging (MRI) or radiography), or in surgery (e.g., by the use of optically visual dye moieties in the immunoimaging agent).

Applicants obtained tumor tissue, snap frozen in the operation hall from unknown patients, which was confirmed as glioblastoma grade IV a by neuropathologist. These tissues served as the experimental sample. Human whole brain tissue (Clontech Laboratories, Palo Alto, USA) served as control sample. Poly-A⁺ RNA prepared from the cells was converted into double-stranded cDNA (dscDNA).

Briefly, the ds-cDNA's from control and disease states were subjected to kinetic re-annealing hybridization during which normalization of transcript abundances and enrichment for differentially expressed transcripts (i.e., subtraction) occurs. Normalized-subtracted ds-cDNAs were cloned into a plasmid vector, a large number of recombinant bacterial clones were picked, and their recombinant inserts were isolated by PCR. High-density cDNA arrays of those PCR products were screened with cDNA probes derived from the original control and disease states. Thus, only clones displaying a significant transcriptional induction and/or repression were sequenced and carried forward for massive expression profiling using a variety of temporal, spatial and disease-related probe sets.

The selected PCR products (fragments of 200-2000 bp in size) from clones showing a significant transcriptional induction and/or repression were sequenced and functionally annotated in AGY's proprietary database structure (See WO01/13105). Because large sequence fragments were utilized in the sequencing step, the data generated has a much higher fidelity and specificity than other approaches, such as SAGE. The resulting sequence information was compared to public databases using the BLAST (blastn) and tblastx algorithm. It was found that PTPζ had a relative expression level approximately 2-4×, and 20 clones were isolated out of 20,000. As one of skilled in the art will appreciate, PTPζ proteins are individually useful as a target for the treatment and/or imaging of brain tumors.

### DISEASE CONDITIONS

The present methods are applicable to brain tumors, particularly glioblastoma. In general, the goals of brain tumor treatments are to remove as many tumor cells as possible, e.g. with surgery, kill as many of the cells left behind after surgery as possible with radiation and/or chemotherapy, and put remaining tumor cells into a nondividing, quiescent state for as long as possible with radiation and chemotherapy. Careful imaging surveillance is a crucial part of medical care, because tumor regrowth requires alteration of current treatment, or, for patients in the observation phase, restarting treatment.

Brain tumors are classified according to the kind of cell from which the tumor seems to originate. Diffuse, fibrillary astrocytomas are the most common type of primary brain tumor in adults. These tumors are divided histopathologically into three grades of malignancy: World Health Organization (WHO) grade II astrocytoma, WHO grade III anaplastic astrocytoma and WHO grade IV glioblastoma multiforme (GBM). WHO grade III astocytomas are the most indolent of the diffuse astrocytoma spectrum. Astrocytomas display a remarkable tendency to infiltrate the surrounding brain, confounding therapeutic attempts at local control. These invasive abilities are often apparent in low-grade as well as high-grade tumors.

Glioblastoma multiforme is the most malignant stage of astrocytoma, with survival times of less than 2 years for most patients. Histologically, these tumors are characterized by dense cellularity, high proliferation indices, endothelial proliferation and focal necrosis. The highly proliferative nature of these lesions likely results from multiple mitogenic effects. One of the hallmarks of GBM is endothelial proliferation. A host of angiogenic growth factors and their receptors are found in GBMs.

There are biologic subsets of astrocytomas, which may reflect the clinical heterogeneity observed in these tumors. These subsets include brain stem gliomas, which are a form of pediatric diffuse, fibrillary astrocytoma that often follow a malignant course. Brain stem GBMs share genetic features with those adult GBMs that affect younger patients. Pleomorphic xanthoastrocytoma (PXA) is a superficial, low-grade astrocytic tumor that predominantly affects young adults. While these tumors have a bizarre histological appearance, they are typically slow-growing tumors that may be amenable to surgical cure. Some PXAs, however, may recur as GBM. Pilocytic astrocytoma is the most common astrocytic tumor of childhood and differs clinically and histopathologically from the diffuse, fibrillary astrocytoma that affects adults. Pilocytic astrocytomas do not have the same genomic alterations as diffuse, fibrillary astrocytomas. Subependymal giant cell astrocytomas (SEGA) are periventricular, low-grade astrocytic tumors that are usually associated with tuberous sclerosis (TS), and are histologically identical to the so-called “candle-gutterings” that line the ventricles of TS patients. Similar to the other tumorous lesions in TS, these are slowly-growing and may be more akin to hamartomas than true neoplasms. Desmoplastic cerebral astrocytoma of infancy (DCAI) and desmoplastic infantile ganglioglioma (DIGG) are large, superficial, usually cystic, benign astrocytomas that affect children in the first year or two of life.

Oligodendrogliomas and oligoastrocytomas (mixed gliomas) are diffuse, usually cerebral tumors that are clinically and biologically most closely related to the diffuse, fibrillary astrocytomas. The tumors, however, are far less common than astrocytomas and have generally better prognoses than the diffuse astrocytomas. Oligodendrogliomas and oligoastrocytomas may progress, either to WHO grade III anaplastic oligodendroglioma or anaplastic oligoastrocytoma, or to WHO grade IV GBM. Thus, the genetic changes that lead to oligodendroglial tumors constitute yet another pathway to GBM.

Ependymomas are a clinically diverse group of gliomas that vary from aggressive intraventricular tumors of children to benign spinal cord tumors in adults. Transitions of ependymoma to GBM are rare. Choroid plexus tumors are also a varied group of tumors that preferentially occur in the ventricular system, ranging from aggressive supratentorial intraventricular tumors of children to benign cerebellopontine angle tumors of adults. Choroid plexus tumors have been reported occasionally in patients with Li-Fraumeni syndrome and von Hippel-Lindau (VHL) disease.

Medulloblastomas are highly malignant, primitive tumors that arise in the posterior fossa, primarily in children. Meningiomas are common intracranial tumors that arise in the meninges and compress the underlying brain. Meningiomas are usually benign, but some “atypical” meningiomas may recur locally, and some meningiomas are frankly malignant and may invade the brain or metastasize. Atypical and malignant meningiomas are not as common as benign meningiomas. Schwannomas are benign tumors that arise on peripheral nerves. Schwannomas may arise on cranial nerves, particularly the vestibular portion of the eighth cranial nerve (vestibular schwannomas, acoustic neuromas) where they present as cerebellopontine angle masses. Hemangioblastomas are tumors of uncertain origin that are composed of endothelial cells, pericytes and so-called stromal cells. These benign tumors most frequently occur in the cerebellum and spinal cord of young adults. Multiple hemangioblastomas are characteristic of von Hippel-Lindau disease (VHL). Hemangiopericytomas (HPCs) are dural tumors which may display locally aggressive behavior and may metastasize. The histogenesis of dural-based hemangiopericytoma (HPC) has long been debated, with some authors classifying it as a distinct entity and others classifying it as a subtype of meningioma.

The symptoms of both primary and metastatic brain tumors depend mainly on the location in the brain and the size of the tumor. Since each area of the brain is responsible for specific functions, the symptoms will vary a great deal. Tumors in the frontal lobe of the brain may cause weakness and paralysis, mood disturbances, difficulty in thinking, confusion and disorientation, and wide emotional mood swings. Parietal lobe tumors may cause seizures, numbness or paralysis, difficulty with handwriting, inability to perform simple mathematical problems, difficulty with certain movements, and loss of the sense of touch. Tumors in the occipital lobe can cause loss of vision in half of each visual field, visual hallucinations, and seizures. Temporal lobe tumors can cause seizures, perceptual and spatial disturbances, and receptive aphasia. If a tumor occurs in the cerebellum, the person may have ataxia, loss of coordination, headaches, and vomiting. Tumors in the hypothalamus may cause emotional changes, and changes in the perception of hot and cold. In addition, hypothalamic tumors may affect growth and nutrition in children. With the exception of the cerebellum, a tumor on one side of the brain causes symptoms and impairment on the opposite side of the body.

Bladder cancer is the second most common malignancy affecting the genitourinary system in the United States. More than 90% of cancers arising in the bladder are transitional cell carcinomas (TCCs)—superficial tumors confined to the epithelial or transitional cell layer of the bladder and are easily treated by transurethral resection. Some TCCs show a mixed pattern with squamous features or a glandular component. Two different configurations of TCCs are seen: papillary and solid. Most tumors are papillary and low grade and do not invade the muscularis propria of the bladder wall. Solid tumors typically are high grade and invasive. A significant correlation exists between grade and prognosis. However, while grade 3 disease is associated with a shorter survival than grade 1, the clinical significance of grade 2 disease is less clear. Tumor staging is based on the degree to which the tumor has invaded into or through the bladder wall. Prognosis correlates with stage but, when controlling for grade, Ta and T1 lesions have similar prognoses.

Breast cancer is the most common malignancy affecting women in North America and Europe. Breast cancer is the second leading cause of cancer death in American women, behind lung cancer. Breast cancer is staged into five different groups. This staging is done in a limited fashion before surgery taking into account the size of the tumor on mammogram and any evidence of spread to other organs that is picked up with other imaging modalities; and it is done definitively after a surgical procedure that removes lymph nodes and allows a pathologist to examine them for signs of cancer.

Invasive ductal and lobular tumors are the most common histologic types of invasive breast cancer (about 90%). Survival rates for patients treated with modified radical mastectomy (simple mastectomy plus lymph node dissection) and for patients treated with breast-conserving surgery (lumpectomy, wide excision, partial mastectomy, or quadrantectomy) plus radiation therapy appear to be identical, at least for the first 20 yr. Most invasive tumors have one or more small areas of intraductal (in situ) cancer; in some studies, tumors with an extensive (>25%) intraductal component (EIC+) within the invasive tumor area and in nearby tissue had a high recurrence rate within the breast after breast-conserving surgery and radiation therapy. However, distant recurrence rates and survival rates after breast-conserving surgery are the same whether the tumor was EIC+ or EIC−. Local control of EIC+ tumors is best achieved by mastectomy or a reexcision of the original tumorous area to rule out multiple foci of remaining tumor. Chemotherapy or endocrine therapy, begun soon after the completion of primary therapy and continued for months or years, delays recurrence in almost all patients and prolongs survival in some.

The most common cancer of the upper respiratory and alimentary tracts is squamous cell carcinoma of the larynx, followed by squamous cell carcinoma of the palatine tonsil and hypopharynx. Head and neck cancers usually remain localized to the head and neck for months to years. Local tissue invasion is followed by metastasis to regional lymph nodes. Distant lymphatic metastases tend to occur late. Hematogenous metastases are usually associated with large or persistent tumors and occur more commonly in immunocompromised patients. Head and neck cancers are traditionally classified clinically according to size and site of the primary neoplasm (T), number and size of metastases to the cervical lymph nodes (N), and evidence of distant metastases (M); several stages are described.

In advanced (most stage II and all stages III and IV) squamous cell carcinoma, a combination of surgery and radiation therapy offers a better chance of cure than does treatment with either alone. Surgery is more effective than radiation therapy and/or chemotherapy in controlling large primary cancers, whereas radiation is more effective in controlling the periphery of the primary lesion and microscopic or nonpalpable metastases. Radiation therapy may be given preoperatively or postoperatively, but the latter is usually preferred.

Adenocarcinoma of the colon and rectum grows slowly, and a long interval elapses before it is large enough to produce symptoms. Early diagnosis depends on routine examination. Symptoms depend on the lesion's location, type, extent, and complications. Primary treatment consists of wide surgical resection of the colon cancer and regional lymphatic drainage after the bowel is prepared. Surgical cure is possible in 70% of patients. The best 5-yr survival rate for cancer limited to the mucosa approaches 90%; with penetration of the muscularis propria, 80%; with positive lymph nodes, 30%. When the patient is an unacceptable surgical risk, some tumors can be controlled locally by electrocoagulation. Preliminary results from studies of adjuvant radiotherapy after curative surgery of rectal (but not colon) cancer suggest that local tumor growth can be controlled, recurrence delayed, and survival improved in patients with limited lymph node involvement.

### PTPζ

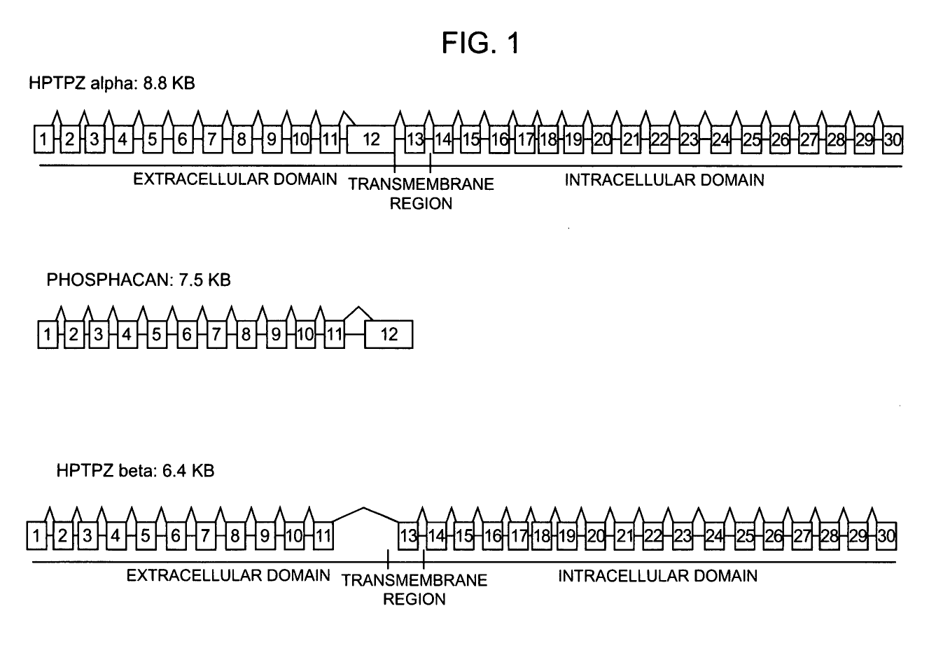

Based on the differential expression described herein, PTPζ was selected as a prime target for selective immuno-therapeutic agents in treating or imaging brain tumors. The complete cDNA sequence encoding PTPζ is provided in SEQ ID NO. 5, and the complete amino acid sequence of PTPζ is provided in SEQ ID NO. 6. Three different splice variants have been described, which include two membrane bound variants (full length: PTPζ- α, and shorter version PTPζ-β) and one secreted form (Phosphacan). See FIG. 1. Isoform PTPζ-α is the full length isoform, which contains the primary amino acid sequence aa 25-2314 of SEQ ID NO. 6 (aa 1-24 are a signal polypeptide). This full length long form of PTPζ is a type I membrane protein. After the signal peptide it contains a carbonic anhydrase like (CAH) and a fibronectin type III like (FN3) domain, followed by a long cysteine free spacer (S) domain. This follows a 860 amino acid long insert domain, which can be glycosylated. After a single transmembrane segment, in the intracellular region it has 2 phosphatase domains, but only the membrane-proximal PTPase domain is catalytically active (Krueger 1992).

In isoform PTPζ-β (sometimes referred to as the short form), aa 755-1614 are missing. Isoform PTPζ-S (phosphacan), is a secreted isoform, which comprises the extracellular domains of PTPζ-α. Northern Blot analysis has shown that the PTPζ is exclusively expressed in the human central nervous system. In mouse embryos, the PTPζ transcript was mainly detected in the ventricular and subventricular zone of the brain and the spinal cord. The same pattern was detected in adult mice. Detailed studies have shown that during rat embryogenesis the two transmembrane splice variants of PTPζ are mainly expressed in glial precursor cells and that the secretory version (Phosphacan) is more abundant in mature astrocytes which have already migrated in the ventricle zone. Applicants have characterized two additional novel slice variants, PTPζ SM1 and PTPζ SM2, which are described in detail below.

As used herein, a compound which specifically binds to human protein tyrosine phosphatase-zeta (PTPζ) is any compound (such as an antibody) that has a binding affinity for any naturally occurring isoform, spice variant, or polymorphism of PTPζ, explicitly including the three splice variants describe herein. For example, the compounds that specifically bind to novel isoforms PTPζ SM1 and PTPζ SM2, described below, are overlapping sets of the compounds that specifically bind to other forms of PTPζ. As one of ordinary skill in the art will appreciate, such “specific” binding compounds (e.g., antibodies) may also bind to other closely related proteins that exhibit significant homology (such as greater than 90% identity, more preferably greater than 95% identity, and most preferably greater than 99% identity) with the amino acid sequence of PTPζ. Such proteins include truncated forms or domains of PTPζ, and recombinantly engineered alterations of PTPζ. For example, a portion of SEQ ID NO. 6 may be engineered to include a non-naturally occurring cysteine for cross-linking to an immunoconjugate protein, as described below.

In general, it is preferred that the antibodies utilized in the compositions and methods of the invention bind to the membrane-bound isoforms of the protein. Of particular interest are antibodies that bind to the ectodomain present in PTPζ-β (residues 26-774 of SEQ ID NO:2), and that recognize the native protein on the surface of living cells. Other useful attributes of the antibodies of the invention include high binding affinity, e.g. of at least about 10 nM K_{D}; and internalization upon binding to living cells. In some embodiments of the invention, the antibody binds to the epitope recognized by one of the 1B9G4; 7A9B5; or 7E4B11 monoclonal antibodies, these antibodies were raised against recombinantly produced and purified extracellular domain of recombinant human PTPζ-β form, including the unique splice junction in BALB/c mice with the protein immunogen in Freund's adjuvant. Spleen cells from immunized mice were fused with a mouse myeloma cell line. The antibodies bind to cells expressing the PTPζ protein on the cell surface. The hybridoma cell lines have been deposited with the American Type Culture Collection, accession number ______.

The amino acid sequence of full length PTPζ consists of 2307 amino acids, as the sequence was deduced by Levy (in which aa 1722-1728 of SEQ ID NO. 2 were missing) (See also U.S. Pat. Nos. 5,604,094, and 6,160,090, fully incorporated herein by reference), or 2314 amino acids as the sequence was deduced by Krueger, et al., (“A human transmembrane protein-tyrosine phosphatase, PTP zeta, is expressed in brain and has an N-terminal receptor domain homologous to carbonic anhydrases” Proc. Nat. Acad. Sci. U.S.A. 89:7417-7421 (1992)). Amino acids 1-24 of SEQ ID NO. 6 are a signal sequence that directs the proper placement of the transmembrane protein. The extracellular domain of the mature PTPζ protein spans amino acids 25-1635 of SEQ ID NO. 6 in the long and secreted forms (this forms the entire secreted form), and amino acids 25-754,1615-1635 in the short isoform. The transmembrane region of the protein spans amino acids 1636-1661 of SEQ ID NO. 6, and the balance of the protein forms the cytoplasmic domain, amino acids 1662-2314.

When raising antibodies to PTPζ, the entire protein (any of the three isoforms) or a portion thereof may be utilized. For instance, the extracellular domain of the long or short form, the entire secreted form, or a portion of extracellular domain may be utilized. Such larger PTPζ proteins and domains may be produced utilizing any suitable recombinant vector/protein production system, such as the baculovirus transfection system outlined below, after being amplified from a fetal brain cDNA library (as available from, e.g., Clontech, Palo Alto, Calif.) or another suitable source. When utilizing an entire protein, or a larger section of the protein, antibodies may be raised by immunizing the production animal with the protein and a suitable adjuvant (e.g., Fruend's, Fruend's complete, oil-in-water emulsions, etc.). In these cases, the PTPζ protein (or a portion thereof) can serve as the PTPζ antigen. When a smaller peptide is utilized, it is advantageous to conjugate the peptide with a larger molecule to make an immunostimulatory conjugate for use as the PTPζ antigen. Commonly utilized conjugate proteins which are commercially available for such use include bovine serum albumin (BSA) and keyhole limpet hemocyanin (KLH). In order to raise antibodies to particular epitopes, peptides derived from the full PTPζ sequence may be utilized. Preferably, one or more 8-30 aa peptide portions of an extracellular domain of PTPζ are utilized, with peptides in the range of 10-20 being a more economical choice. Custom-synthesized peptides in this range are available from a multitude of vendors, and can be order conjugated to KLH or BSA. Alternatively, peptides in excess of 30 amino acids may be synthesized by solid-phase methods, or may be recombinantly produced in a suitable recombinant protein production system. In order to ensure proper protein glycosylation and processing, an animal cell system (e.g., Sf9 or other insect cells, CHO or other mammalian cells) is preferred. Other information useful in designing an antigen for the production of antibodies to PTPζ, including glycosylation sites, is provided in SEQ ID NO. 6.

The extracellular domain of human PTPζ is known to bind to tenascin-C, tenascin-R, pleiotrophin (NM_{—}002825), midkine (NM_{—}002391), FGF-2 (XM_{—}00366), Nr-CAM (NM_{—}005010), L1/Ng-CAM, contactin (NM_{—}001843), N-CAM (XM_{—}006332), and axonin-1NM_{—}005076.) The first 5 of these molecules are either components of the extracellular matrix in gliomas or are soluble factors known to be present in gliomas, and the latter 4 are neuronal surface molecules. The binding of PTPζ to these molecules may play a significant role in the oncogenesis and growth of neoplastic cells in the brain. Thus, in alternative embodiments of the compositions and methods of the invention, antibody moieties are utilized which bind to PTPζ at a site on the protein which alters the binding of an extracellular ligand molecule to PTPζ. Such PTPζ activity altering antibodies may be utilized in therapeutic compositions in an unconjugated form (e.g., the antibody in an acceptable pharmaceutical carrier), or may be conjugated to either a therapeutic moiety (creating a double-acting therapeutic agent) or an imaging moiety (creating a duel therapeutic/imaging agent).

Selection of antibodies that alter (enhance or inhibit) the binding of a ligand to PTPζ may be accomplished by a straightforward binding inhibition/enhancement assay. According to standard techniques, the binding of a labeled (e.g., fluorescently or enzyme-labeled) antibody to PTPζ, which has been immobilized in a microtiter well, is assayed in both the presence and absence of the ligand. The change in binding is indicative of either an enhancer (increased binding) or competitive inhibitor (decreased binding) relationship between the antibody and the ligand. Such assays may be carried out in high-throughput formats (e.g., 384 well plate formats, in robotic systems) for the automated selection of monoclonal antibody candidates for use as PTPζ ligand-binding inhibitors or enhancers.

In addition, antibodies that are useful for altering the function of PTPζ may be assayed in functional formats, such as the HUVEC tube assay and the cell migration assay described below. Thus, antibodies that exhibit the appropriate anti-PTPζ activity may be selected without direct knowledge of the biomolecular role of PTPζ.

### Novel PTPζ Splice Variants PTPζ SM1 and PTPζ SM2

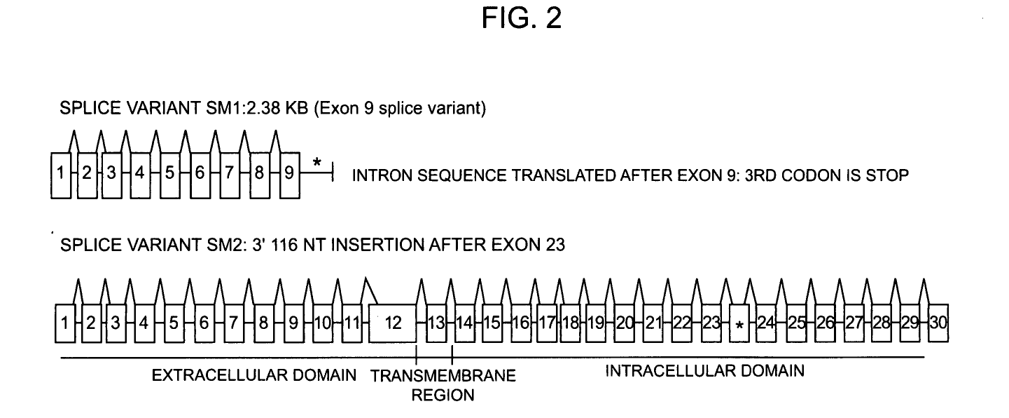

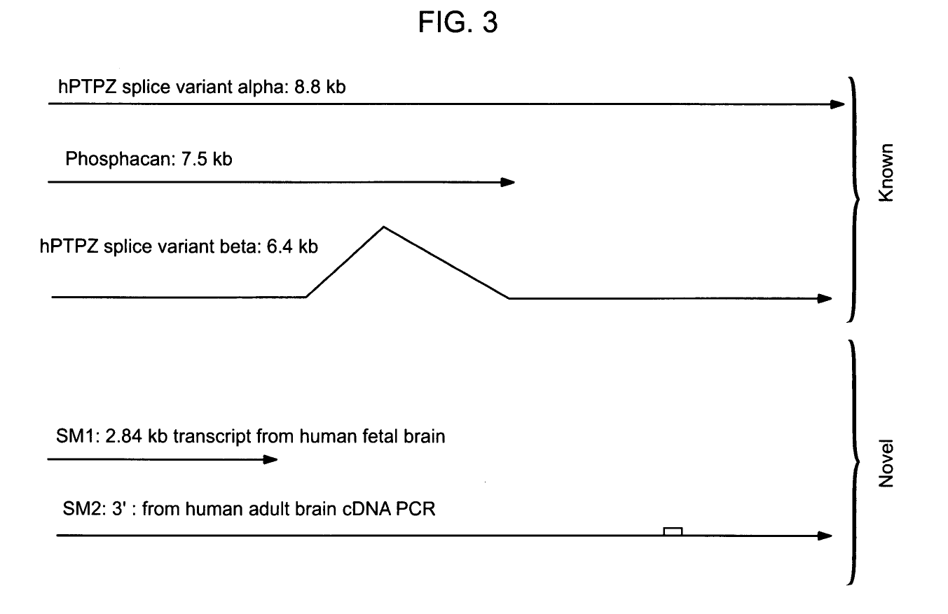

In addition to the known variants of PTPζ for use in the invention, applicants have identified two novel splice variant isoforms of PTPζ, SM1 and SM2, from their clone libraries, see FIG. 2. These novel isoforms, PTPζ SM1 and PTPζ SM2, differ in structure from the three known isoforms heretofore disclosed, as is illustrated in FIG. 3. As only cDNA sequences for the known splice variants had been previously disclosed, rather than the full gene sequence, applicants verified the location of the novel sequences by comparison of the known splice variant sequences and the novel sequences with a publicly available genomic sequence database.

The protein PTPζ SM1 (amino acid sequence SEQ ID NO. 2, cDNA sequence SEQ ID NO. 1) comprises the amino acids encoded by the first nine exons of PTPζ-α, with three unique additional carboxy terminal amino acids, see FIG. 2. These are encoded by additional 3′ mRNA sequence (nucleotides 1262-1272 of SEQ ID NO. 1) from the intron of the gene between exons nine and ten. The PTPζ SM1 clone was isolated from a human fetal brain cDNA library, and has been shown to be expressed in several human glioblastoma cell lines. Expression of the SM1 splice variant has also been confirmed in primary brain tumor samples. The protein comprises only extracellular domains of PTPζ, and is expected to be secreted by the cell. Thus, PTPζ SM1 may serve a cell signaling or messenger function, and may have bind to a receptor on the surface of cells which are associated with or part of central nervous system tissues. Thus, antibodies specific for PTPζ SM1, and not specific for the other splicing isoforms of PTPζ, may be especially efficacious in the brain tumor therapeutic or imaging compositions of the invention. The PTPζ SM1 protein mainly comprises the carbonic anhydrase-like domain which has been identified in PTPζ α.

The protein PTPζ SM2 (amino acid sequence SEQ ID NO. 4) comprises the amino acids encoded by all exons of PTPζ-α, plus a 116 nucleotide “extra” exon, in the correct reading frame, between exons 23 and 24 (nucleotides 6229-6345 of SEQ ID NO. 3). This extra exon, designated exon 23a, contains a portion of the intron sequence between exons 23 and 24 of the PTPζ gene. PTPζ SM2 expression has been verified in several human glioblastoma cell lines, and has also been confirmed in primary brain tumor samples. As PTPζ SM2 comprises all the domains of PTPζ α, the protein is expected to be membrane-bound. The extra exon lies within the cytoplasmic domain of the protein, and thus may alter the protein tyrosine phosphatase function of PTPζ SM2.

A novel splicing variant PTPζ protein having an amino acid sequence which includes the amino acid sequence of PTPζ SM1 (SEQ ID NO. 2) or PTPζ SM2 (SEQ. ID NO. 4) may be produced by recombinant techniques known in the art utilizing any suitable vector, in any suitable host cell. The term “vector” is intended to include any physical or biochemical vehicle containing nucleic acid polymers of interest, by which those nucleic acid polymers are transferred into a host cell, thereby transfecting that cell with the introduced nucleic acid polymers. The transfected nucleic acid sequence preferably contains a control sequence, such as a promoter sequence, suitable for transcription of the nucleic acid sequence in the host cell. Examples of vectors include DNA plasmids, viruses, liposomes, particle gun pellets, and transfection vectors known to those of skill in the molecular biology arts. The term “host cell” is intended to mean the target cell for vector transformation, in which the transferred nucleic acid polymer will be replicated and/or expressed. Although bacterial cells may be suitable for production of the proteins for antibody production or structural study purposes, eukaryotic cell hosts are preferred for production of the protein for functional assays or therapeutic purposes. Preferred eukaryotic cell hosts include insect cell lines (e.g, Sf9, Sf21, or High Five™ cell lines), and mammalian cell lines (e.g., HeLa, CHO-K1, COS-7, COS-1, HEK293, HEPG2, Jurkat, MDCK, PAE, PC-12, and other acceptable mammalian cell lines). Thus, the invention also provides vectors incorporating a nucleic acid sequence encoding PTPζ SM1 or PTPζ SM2, as well as host cells which express the proteins.

The invention also provides nucleic acid polymers encoding the PTPζ splice variants SM1 or SM2. These nucleic acid polymers most preferably comprises a nucleic acid sequence of SEQ. ID NO. 1 or SEQ ID NO. 3, or the predictable variants thereof which one of ordinary skill of the art could derive using the degeneracy of the genetic code. Such nucleic acid polymers are useful for the production of PTPζ SM1 or PTPζ SM2 by recombinant methods, as described above.

The invention also encompasses nucleic acid probes or primers which hybridize to the mRNA encoding PTPζ splice variants SM1 or SM2, but not mRNA encoding other known splice variants of PTPζ. Such probes or primers provided by the invention are preferably able to hybridize with SEQ. ID NO. 1 or SEQ. ID NO. 3 (or their complements) under stringent conditions (e.g., 0.5× to 2× SSC buffer, 0.1% SDS, and a temperature of 55-65° C.), but do not hybridize to SEQ ID NO. 5 (or its complement) under the same conditions. These PTPζ SM1 or PTPζ SM2 coding sequence specific probes are preferably from about 16 to about 40 nucleotides in length, more preferably from about 18 nucleotides to about 30 nucleotides in length. However, probes may be of a smaller size, preferably from about 8 to about 15 nucleotides in length, if two ore more probes are hybridized to adjacent sequences, so that terminal nucleic acid base-stacking interactions may stabilize their hybridization. In preferred embodiments of PTPζ SM1 specific nucleic acid probes, the probes hybridize at or near the novel splice site at the 3′ end of exon 9, or its complement. In preferred embodiments of PTPζ SM2 specific probes, the probes hybridize at or adjacent to a location selected from: the novel splice site at the 3′ end of exon 23, at least a portion of the novel exon 23a, the novel splice site at the 5′ end of exon 24, or the complement of any one of these.

Because PTPζ SM1 and PTPζ SM2 have been shown to be expressed in glioblastoma cell lines and primary tumors, the level of the expression of these splice variants may be useful for staging or characterizing glioblastoma cells. Such cells may be extracted, for instance, from a primary tumor. Thus, the invention provides for the monitoring of the relative expression level of PTPζ SM1 or PTPζ SM2, or both, in relation to each other or to one or more of the known PTPζ splice variants. In one preferred embodiment, the level of expression of PTPζ SM1 is compare to at least one other splice variant selected from PTPζ SM2, PTPζ α, PTPζ β, and phosphacan. In another preferred embodiment, the level of expression of PTPζ SM2 is compare to at least one other splice variant selected from PTPζ SM1, PTPζ α, PTPζ β, and phosphacan. Such comparison may be made in either a qualitative or quantitative manner. One means for comparison is by hybridizing splice-variant specific nucleic acid probes to a sample of nucleic acids (which may be amplified) obtained from brain tumor cells. Alternatively, the expression level of the splice variants may be deduced by the amplification of splice variant nucleic acid sequences, and the analysis of the size of those amplified products using methods known in the art. In another alternative embodiment, protein levels may be studied utilizing splice-variant specific antibodies in either sandwich immunoassay or in-situ staining formats. Various expression level assay techniques are known to those of skill in the molecular biological arts, and thus the specific techniques mentioned above should be considered merely exemplary.

### Antibodies for Use in the Antibody-Therapeutics Methods of the Invention

Generally, as the term is utilized in the specification, “antibody” or “antibody moiety” is intended to include any polypeptide chain-containing molecular structure that has a specific shape which fits to and recognizes an epitope, where one or more non-covalent binding interactions stabilize the complex between the molecular structure and the epitope. Antibodies that bind specifically to a PTPζ isoform are referred to as α(PTPζ). The specific or selective fit of a given structure and its specific epitope is sometimes referred to as a “lock and key” fit. The archetypal antibody molecule is the immunoglobulin, and all types of immunoglobulins (IgG, IgM, IgA, IgE, IgD, etc.), from all sources (e.g., human, rodent, rabbit, cow, sheep, pig, dog, other mammal, chicken, turkey, emu, other avians, etc.) are considered to be “antibodies.” Antibodies utilized in the present invention may be polyclonal antibodies, although monoclonal antibodies are preferred because they may be reproduced by cell culture or recombinantly, and may be modified to reduce their antigenicity.

Polyclonal antibodies may be raised by a standard protocol by injecting a production animal with an antigenic composition, formulated as described above. See, e.g., Harlow and Lane, Antibodies: A Laboratory Manual, Cold Spring Harbor Laboratory, 1988. In one such technique, an antigen comprising an antigenic portion of the target polypeptide is initially injected into any of a wide variety of mammals (e.g., mice, rats, rabbits, sheep or goats). Alternatively, in order to generate antibodies to relatively short peptide portions of the brain tumor protein target (see discussion above), a superior immune response may be elicited if the polypeptide is joined to a carrier protein, such as ovalbumin, BSA or KLH. The peptide-conjugate is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. The immortal cell line utilized is preferably selected to be deficient in enzymes necessary for the utilization of certain nutrients. Many such cell lines (such as myelomas) are known to those skilled in the art, and include, for example: thymidine kinase (TK) or hypoxanthine-guanine phosphoribosyl transferase (HGPRT). These deficiencies allow selection for fused cells according to their ability to grow on, for example, hypoxanthine aminopterinthymidine medium (HAT).

Preferably, the immortal fusion partners utilized are derived from a line that does not secrete immunoglobulin. The resulting fused cells, or hybridomas, are cultured under conditions that allow for the survival of fused, but not unfused, cells and the resulting colonies screened for the production of the desired monoclonal antibodies. Colonies producing such antibodies are cloned, expanded, and grown so as to produce large quantities of antibody, see Kohler and Milstein, 1975 Nature 256:495 (the disclosures of which are hereby incorporated by reference).

Large quantities of monoclonal antibodies from the secreting hybridomas may then be produced by injecting the clones into the peritoneal cavity of mice and harvesting the ascites fluid therefrom. The mice, preferably primed with pristine, or some other tumor-promoter, and immunosuppressed chemically or by irradiation, may be any of various suitable strains known to those in the art. The ascites fluid is harvested from the mice and the monoclonal antibody purified therefrom, for example, by CM Sepharose column or other chromatographic means. Alternatively, the hybridomas may be cultured in vitro or as suspension cultures. Batch, continuous culture, or other suitable culture processes may be utilized. Monoclonal antibodies are then recovered from the culture medium or supernatant.

Preferred monoclonal antibodies are those described here, e.g. 1B9G4; 7A9B5; or 7E4B11, or antibodies that bind the epitopes recognized by 1B9G4; 7A9B5; or 7E4B11. Alternatively, monoclonal antibodies against various isoforms are currently available from commercial sources. For instance, a non-exclusive list of available commercial antibodies includes: for PTPζ-α and PTPζ-β, from BD Transduction Labs, mouse anti-human MAb (WB, IH, IF), denominated “R20720” and from Chemicon, mouse anti-human MAb (WB, IH, IP), denominated “MAB5210”, which recognizes both of the transmembrane isoforms, and also recognizes the soluble isoform (phosphacan, PTPζ-S).

Antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell.). When antibody gene synthesis is induced in the transfected host, the heavy and light chain proteins self-assemble to produce active antibodies that can be detected by screening with the antigen or immunogen.

Preferably, recombinant antibodies are produced in a recombinant protein production system which correctly glycosylates and processes the immunoglobulin chains, such as insect or mammalian cells. An advantage to using insect cells which utilize recombinant baculoviruses for the production of antibodies for use in the present invention is that the baculovirus system allows production of mutant antibodies much more rapidly than stably transfected mammalian cell lines. In addition, insect cells have been shown to correctly process and glycosylate eukaryotic proteins, which prokaryotic cells do not. Finally, the baculovirus expression of foreign protein has been shown to constitute as much as 50-75% of the total cellular protein late in viral infection, making this system an excellent means of producing milligram quantities of the recombinant antibodies.

The use of the baculovirus Autographia californica nuclear polyhedrosis virus (AcNPV) and recombinant viral stocks in Spodoptera frugiperda (Sf9) cells to prepare large quantities of protein has been described by Smith et al. (1985), Summers and Smith (1987). A preferred method of preparing recombinant antibodies is through the expression of DNA encoding recombinant antibody (produced by screening, as above, or by protein engineering to include more human-like domains, as discussed below) via the baculoviral expression system in Sf9 insect cells. Production of recombinant proteins in Sf9 cells is well known in the art, and one of ordinary skill would be able to select from a number of acceptable protocols (e.g., that described in U.S. Pat. No. 6,603,905).

It should be noted that antibodies which have a reduced propensity to induce a violent or detrimental immune response in humans (such as anaphylactic shock), and which also exhibit a reduced propensity for priming an immune response which would prevent repeated dosage with the antibody therapeutic or imaging agent (e.g., the human-anti-murine-antibody “HAMA” response), are preferred for use in the invention. These antibodies are preferred for all administrative routes, including intrathecal administration. Even through the brain is relatively isolated in the cranial cavity, behind the blood brain barrier, an immune response still can occur in the form of increased leukocyte infiltration, and inflammation. Although some increased immune response against the tumor is desirable, the concurrent binding and inactivation of the therapeutic or imaging agent generally outweighs this benefit. Thus, humanized, chimeric, or xenogenic human antibodies, which produce less of an immune response when administered to humans, are preferred for use in the present invention.

Chimeric antibodies may be made by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference). Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward in practice. See, e.g., U.S. Pat. No. 6,187,287, incorporated fully herein by reference.

Alternatively, polyclonal or monoclonal antibodies may be produced from animals which have been genetically altered to produce human immunoglobulins, such as the Abgenix XenoMouse or the Medarex HuMAb® technology. The transgenic animal may be produced by initially producing a “knock-out” animal which does not produce the animal's natural antibodies, and stably transforming the animal with a human antibody locus (e.g., by the use of a human artificial chromosome). Only human antibodies are then made by the animal. Techniques for generating such animals, and deriving antibodies therefrom, are described in U.S. Pat. Nos. 6,162,963 and 6,150,584, incorporated fully herein by reference. Such fully human xenogenic antibodies are a preferred antibody for use in the methods and compositions of the present invention.

Alternatively, single chain antibodies (Fv, as described below) can be produced from phage libraries containing human variable regions. See U.S. Pat. No. 6,174,708, incorporated fully herein by reference. Also see Kuan, C. T., Reist, C. J., Foulon, C. F., Lorimer, I. A., Archer, G., Pegram, C. N., Pastan, I., Zalutsky, M. R., and Bigner, D. D. (1999). 125I-labeled anti-epidermal growth factor receptor-viii single-chain Fv exhibits specific and high-level targeting of glioma xenografts. Clin Cancer Res. 5, 1539-49; Lorimer, I. A., Keppler-Hafkemeyer, A., Beers, R. A., Pegram, C. N., Bigner, D. D., and Pastan, I. (1996). Recombinant immunotoxins specific for a mutant epidermal growth factor receptor: targeting with a single chain antibody variable domain isolated by phage display. Proc. Nat. Acad. Sci. USA 93, 14815-20; Pastan, I. H., Archer, G. E., McLendon, R. E., Friedman, H. S., Fuchs, H. E., Wang, Q. C., Pai, L. H., Herndon, J., and Bigner, D. D. (1995). Intrathecal administration of single-chain immunotoxin, LMB-7 [B3(Fv)- PE38], produces cures of carcinomatous meningitis in a rat model. Proc Natl. Acad. Sci USA 92, 2765-9, all of which are incorporated by reference fully herein.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif.

Fv fragments are heterodimers of the variable heavy chain domain (V_{H}) and the variable light chain domain (V_{L}). The heterodimers of heavy and light chain domains that occur in whole IgG, for example, are connected by a disulfide bond. Recombinant Fvs in which V_{H} and V_{L} are connected by a peptide linker are typically stable, see, for example, Huston et al., Proc. Natl. Acad, Sci. USA 85:5879-5883 (1988) and Bird et al., Science 242:423-426 (1988), both fully incorporated herein, by reference. These are single chain Fvs which have been found to retain specificity and affinity and have been shown to be useful for imaging tumors and to make recombinant immunotoxins for tumor therapy. However, researchers have bound that some of the single chain Fvs have a reduced affinity for antigen and the peptide linker can interfere with binding. Improved Fv's have been also been made which comprise stabilizing disulfide bonds between the V_{H} and V_{L} regions, as described in U.S. Pat. No. 6,147,203, incorporated fully herein by reference. Any of these minimal antibodies may be utilized in the present invention, and those which are humanized to avoid HAMA reactions are preferred for use in embodiments of the invention.

In addition, derivatized immunoglobulins with added chemical linkers, detectable moieties, e.g. fluorescent dyes, enzymes, substrates, chemiluminescent moieties, or specific binding moieties such as streptavidin, avidin, or biotin may be utilized in the methods and compositions of the present invention. For convenience, the term “antibody” or “antibody moiety” will be used throughout to generally refer to molecules which specifically bind to an epitope of the tumor protein targets, although the term will encompass all immunoglobulins, derivatives, fragments, recombinant or engineered immunoglobulins, and modified immunoglobulins, as described above.

Candidate antibodies can be tested for activity by any suitable standard means. As a first screen, the antibodies may be tested for binding against the tumor protein target antigen utilized to produce them, or against the entire brain tumor protein target extracellular domain or protein. As a second screen, antibody candidates may be tested for binding to an appropriate glioblastoma cell line (i.e., one which approximates primary tumor brain tumor protein target expression), or to primary tumor tissue samples. For these screens, the candidate antibody may be labeled for detection (e.g., with fluorescein or another fluorescent moiety, or with an enzyme such as horseradish peroxidase). After selective binding to the tumor protein target is established, the candidate antibody, or an antibody conjugate produced as described below, may be tested for appropriate activity (i.e., the ability to decrease tumor cell growth and/or to aid in visualizing tumor cells) in an in vivo model, such as an appropriate glioblastoma cell line, or in a mouse or rat human brain tumor model, as described below.

### General Functional Assay Methods for Antibodies for Use in the Invention

In addition to the specific binding assays and protein-specific functional assays described for individual proteins above, antibodies which are useful for altering the function of PTPζ may be assayed in functional formats, such as glioblastoma cell culture or mouse/rat CNS tumor model studies. In glioblastoma cell models of activity, expression of the protein is first verified in the particular cell strain to be used. If necessary, the cell line may be stably transfected with a coding sequence of the protein under the control of an appropriate constituent promoter, in order to express the protein at a level comparable to that found in primary tumors. The ability of the glioblastoma cells to survive in the presence of the candidate function-altering anti-protein antibody is then determined. In addition to cell-survival assays, cell migration assays, as described below in Example 1, may be utilized to determine the effect of the candidate antibody therapeutic agent on the tumor-like behavior of the cells. Alternatively, if the brain tumor protein target is involved in angiogenesis, or endothelial cell sprouting assays such as described in Example 2 may be utilized to determine the ability of the candidate antibody therapeutic to inhibit vascular neogenesis, an important function in tumor biology.

Similarly, in vivo models for human brain tumors, particularly nude mice/SCID mice model or rat models, have been described [Antunes, L., Angioi-Duprez, K. S., Bracard, S. R., Klein-Monhoven, N. A., Le Faou, A. E., Duprez, A. M., and Plenat, F. M. (2000). Analysis of tissue chimerism in nude mouse brain and abdominal xenograft models of human glioblastoma multiforme: what does it tell us about the models and about glioblastoma biology and therapy. J Histochem Cytochem 48, 847-58; Price, A., Shi, Q., Morris, D., Wilcox, M. E., Brasher, P. M., Rewcastle, N. B., Shalinsky, D., Zou, H., Appelt, K., Johnston, R. N., Yong, V. W., Edwards, D., and Forsyth, P. (1999). Marked inhibition of tumor growth in a malignant glioma tumor model by a novel synthetic matrix metalloproteinase inhibitor AG3340. Clin Cancer Res 5, 845-54; and Senner, V., Sturm, A., Hoess, N., Wassmann, H., and Paulus, W. (2000). In vivo glioma model enabling regulated gene expression. Acta Neuropathol (Berl) 99, 603-8.] Once correct expression of the protein in the tumor model is verified, the effect of the candidate anti-protein antibodies on the tumor masses in these models can be evaluated, wherein the ability of the anti-protein antibody candidates to alter protein activity is indicated by a decrease in tumor growth or a reduction in the tumor mass. Thus, antibodies that exhibit the appropriate anti-tumor effect may be selected without direct knowledge of the particular biomolecular role of the protein in oncogenesis.

### Therapeutic and Imaging Moieties, and Methods for Conjugating them with Anti-PTPζ Antibodies to Use in the Compositions and Methods of the Invention

As described above and in the Examples, the anti-PTPζ antibodies have utility without conjugation, acting to inhibit the growth of tumor cells. However, the cytotoxic effect is enhanced by conjugation with a cytotoxic moiety; and for imaging purposes it is desirable to conjugate antibodies to an imaging moiety.

As used herein, “cytotoxic moiety” (C) simply means a moiety which inhibits cell growth or promotes cell death when proximate to or absorbed by the cell. Suitable cytotoxic moieties in this regard include radioactive isotopes (radionuclides), chemotoxic agents such as differentiation inducers and small chemotoxic drugs, toxin proteins such as saporin, and derivatives thereof. As utilized herein, “imaging moiety” (I) means a moiety which can be utilized to increase contrast between a tumor and the surrounding healthy tissue in a visualization technique (e.g., radiography, positron-emission tomography, magnetic resonance imaging, direct or indirect visual inspection). Thus, suitable imaging moieties include radiography moieties (e.g. heavy metals and radiation emitting moieties), positron emitting moieties, magnetic resonance contrast moieties, and optically visible moieties (e.g., fluorescent or visible-spectrum dyes, visible particles, etc.). It will be appreciated by one of ordinary skill that some overlap exists between what is a therapeutic moiety and what is an imaging moiety. For instance ²¹²Pb and ²¹²Bi are both useful radioisotopes for therapeutic compositions, but are also electron-dense, and thus provide contrast for X-ray radiographic imaging techniques, and can also be utilized in scintillation imaging techniques.

In general, therapeutic or imaging agents may be conjugated to the anti-PTPζ moiety by any suitable technique, with appropriate consideration of the need for pharmokinetic stability and reduced overall toxicity to the patient. A therapeutic agent may be coupled to a suitable antibody moiety either directly or indirectly (e.g. via a linker group). A direct reaction between an agent and an antibody is possible when each possesses a functional group capable of reacting with the other. For example, a nucleophilic group, such as an amino or sulfhydryl group, may be capable of reacting with a carbonyl-containing group, such as an anhydride or an acid halide, or with an alkyl group containing a good leaving group (e.g., a halide). Alternatively, a suitable chemical linker group may be used. A linker group can function as a spacer to distance an antibody from an agent in order to avoid interference with binding capabilities. A linker group can also serve to increase the chemical reactivity of a substituent on a moiety or an antibody, and thus increase the coupling efficiency. An increase in chemical reactivity may also facilitate the use of moieties, or functional groups on moieties, which otherwise would not be possible.

Suitable linkage chemistries include maleimidyl linkers and alkyl halide linkers (which react with a sulfhydryl on the antibody moiety) and succinimidyl linkers (which react with a primary amine on the antibody moiety). Several primary amine and sulfhydryl groups are present on immunoglobulins, and additional groups may be designed into recombinant immunoglobulin molecules. It will be evident to those skilled in the art that a variety of bifunctional or polyfunctional reagents, both homo- and hetero-functional (such as those described in the catalog of the Pierce Chemical Co., Rockford, Ill.), may be employed as a linker group. Coupling may be effected, for example, through amino groups, carboxyl groups, sulfhydryl groups or oxidized carbohydrate residues. There are numerous references describing such methodology, e.g., U.S. Pat. No. 4,671,958. As an alternative coupling method, cytotoxic or imaging moieties may be coupled to the anti-T_{BT} antibody moiety through a an oxidized carbohydrate group at a glycosylation site, as described in U.S. Pat. Nos. 5,057,313 and 5,156,840. Yet another alternative method of coupling the antibody moiety to the cytotoxic or imaging moiety is by the use of a non-covalent binding pair, such as streptavidin/biotin, or avidin/biotin. In these embodiments, one member of the pair is covalently coupled to the antibody moiety and the other member of the binding pair is covalently coupled to the cytotoxic or imaging moiety.

Where a cytotoxic moiety is more potent when free from the antibody portion of the immunoconjugates of the present invention, it may be desirable to use a linker group which is cleavable during or upon internalization into a cell, or which is gradually cleavable over time in the extracellular environment. A number of different cleavable linker groups have been described. The mechanisms for the intracellular release of a cytotoxic moiety agent from these linker groups include cleavage by reduction of a disulfide bond (e.g., U.S. Pat. No. 4,489,710), by irradiation of a photolabile bond (e.g., U.S. Pat. No. 4,625,014), by hydrolysis of derivatized amino acid side chains (e.g., U.S. Pat. No. 4,638,045), by serum complement-mediated hydrolysis (e.g., U.S. Pat. No. 4,671,958), and acid-catalyzed hydrolysis (e.g., U.S. Pat. No. 4,569,789).

It may be desirable to couple more than one cytotoxic and/or imaging moiety to an antibody. By poly-derivatizing the antibody, several cytotoxic strategies may be simultaneously implemented, an antibody may be made useful as a contrasting agent for several visualization techniques, or a therapeutic antibody may be labeled for tracking by a visualization technique. In one embodiment, multiple molecules of an imaging or cytotoxic moiety are coupled to one antibody molecule. In another embodiment, more than one type of moiety may be coupled to one antibody. Regardless of the particular embodiment, immunoconjugates with more than one moiety may be prepared in a variety of ways. For example, more than one moiety may be coupled directly to an antibody molecule, or linkers that provide multiple sites for attachment (e.g., dendrimers) can be used. Alternatively, a carrier with the capacity to hold more than one cytotoxic or imaging moiety can be used.

A carrier may bear the agents in a variety of ways, including covalent bonding either directly or via a linker group, and non-covalent associations. Suitable covalent-bond carriers include proteins such as albumins (e.g., U.S. Pat. No. 4,507,234), peptides, and polysaccharides such as aminodextran (e.g., U.S. Pat. No. 4,699,784), each of which have multiple sites for the attachment of moieties. A carrier may also bear an agent by non-covalent associations, such as non-covalent bonding or by encapsulation, such as within a liposome vesicle (e.g., U.S. Pat. Nos. 4,429,008 and 4,873,088). Encapsulation carriers are especially useful for imaging moiety conjugation to antibody moieties for use in the invention, as a sufficient amount of the imaging moiety (dye, magnetic resonance contrast reagent, etc.) for detection may be more easily associated with the antibody moiety. In addition, encapsulation carriers are also useful in chemotoxic therapeutic embodiments, as they can allow the therapeutic compositions to gradually release a chemotoxic moiety over time while concentrating it in the vicinity of the tumor cells.

Carriers and linkers specific for radionuclide agents (both for use as cytotoxic moieties or positron-emission imaging moieties) include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis. Such chelation carriers are also useful for magnetic spin contrast ions for use in magnetic resonance imaging tumor visualization methods, and for the chelation of heavy metal ions for use in radiographic visualization methods.

Preferred radionuclides for use as cytotoxic moieties are radionuclides which are suitable for pharmacological administration. Such radionuclides include ¹²³I, ¹²⁵I, ¹³¹I, ⁹⁰Y, ²¹¹At, ⁶⁷Cu, ¹⁸⁶Re, ¹⁸⁸Re, ²¹²Pb, and ²¹²Bi. Iodine and astatine isotopes are more preferred radionuclides for use in the therapeutic compositions of the present invention, as a large body of literature has been accumulated regarding their use. ¹³¹I is particularly preferred, as are other β-radiation emitting nuclides, which have an effective range of several millimeters. ¹²³I, ¹²⁵I, ¹³¹I, or ²¹¹At may be conjugated to antibody moieties for use in the compositions and methods utilizing any of several known conjugation reagents, including Iodogen, N-succinimidyl 3-[²¹¹At]astatobenzoate, N-succinimidyl 3-[¹³¹I]iodobenzoate (SIB), and, N-succinimidyl 5-[¹³¹I]iodob-3-pyridinecarboxylate (SIPC). Any iodine isotope may be utilized in the recited iodo-reagents. Other radionuclides may be conjugated to anti-T_{BT} antibody moieties by suitable chelation agents known to those of skill in the nuclear medicine arts.

Preferred chemotoxic agents include small-molecule drugs such as carboplatin, cisplatin, vincristine, taxanes such as paclitaxel and doceltaxel, hydroxyurea, gemcitabine, vinorelbine, irinotecan, tirapazamine, matrilysin, methotrexate, pyrimidine and purine analogs, and other suitable small toxins known in the art. Preferred chemotoxin differentiation inducers include phorbol esters and butyric acid. Chemotoxic moieties may be directly conjugated to the antibody via a chemical linker, or may encapsulated in a carrier, which is in turn coupled to the antibody.

Preferred toxin proteins for use as cytotoxic moieties include ricins A and B, abrin, diphtheria toxin, bryodin 1 and 2, momordin, trichokirin, cholera toxin, gelonin, Pseudomonas exotoxin, Shigella toxin, pokeweed antiviral protein, saporin, and other toxin proteins known in the medicinal biochemistry arts. As these toxin agents may elicit undesirable immune responses in the patient, especially if injected intravascularly, it is preferred that they be encapsulated in a carrier for coupling to the antibody.

Preferred radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the compositions and methods of the invention. Examples of such compositions, which may be utilized for x-ray radiography are described in U.S. Pat. No. 5,709,846, incorporated fully herein by reference. Such moieties may be conjugated to the antibody through an acceptable chemical linker or chelation carrier. In addition, radionuclides that emit radiation capable of penetrating the skull may be useful for scintillation imaging techniques. Suitable radionuclides for conjugation include ⁹⁹Tc, ¹¹¹In, and ⁶⁷Ga. Positron emitting moieties for use in the present invention include ¹⁸F, which can be easily conjugated by a fluorination reaction with the antibody according to the method described in U.S. Pat. No. 6,187,284.

Preferred magnetic resonance contrast moieties include chelates of chromium(III), manganese(II), iron(II), nickel(II), copper(II), praseodymium(III), neodymium(III), samarium(III) and ytterbium(III) ion. Because of their very strong magnetic moment, the gadolinium(III), terbium(III), dysprosium(III), holmium(III), erbium(III), and iron(III) ions are especially preferred. Examples of such chelates, suitable for magnetic resonance spin imaging, are described in U.S. Pat. No. 5,733,522, incorporated fully herein by reference. Nuclear spin contrast chelates may be conjugated to the antibodies through a suitable chemical linker.

Optically visible moieties for use as imaging moieties include fluorescent dyes, or visible-spectrum dyes, visible particles, and other visible labeling moieties. Fluorescent dyes such as fluorescein, coumarin, rhodamine, bodipy Texas red, and cyanine dyes, are useful when sufficient excitation energy can be provided to the site to be inspected visually. Endoscopic visualization procedures may be more compatible with the use of such labels. For many procedures where imaging agents are useful, such as during an operation to resect a brain tumor, visible spectrum dyes are preferred. Acceptable dyes include FDA-approved food dyes and colors, which are non-toxic, although pharmaceutically acceptable dyes which have been approved for internal administration are preferred. In preferred embodiments, such dyes are encapsulated in carrier moieties, which are in turn conjugated to the anti-T_{BT} antibody. Alternatively, visible particles, such as colloidal gold particles or latex particles, may be coupled to the anti-T_{BT} antibody moiety via a suitable chemical linker.

### Delivery of Therapeutic and Imaging Agents to the Patient

The mode of delivery of the antibody to a patient will depend on the specific type of tumor that is being treated. For many embodiments, direct antitumor injection may be utilized, or systemic injection, e.g. intra-vascular injection, etc. Where the tumor is a brain tumor, special considerations may arise to bring the therapeutic or imaging composition across the blood brain barrier (BBB). A first strategy for drug delivery through the BBB entails disruption of the BBB, either by osmotic means such as mannitol or leukotrienes, or biochemically by the use of vasoactive substances such as bradykinin. The potential for using BBB opening to target specific agents to brain tumors is also an option. In preferred embodiments, a BBB disrupting agent is co-administered with the therapeutic or imaging compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. However, the best current strategy for drug delivery behind the BBB is by intrathecal delivery of therapeutics or imaging agents directly to the cranium, as through an Ommaya reservoir.

For administration to any of the tumors of interest, the antibody-therapeutic or antibody-imaging agent will generally be mixed, prior to administration, with a non-toxic, pharmaceutically acceptable carrier substance. Usually, this will be an aqueous solution, such as normal saline or phosphate-buffered saline (PBS), Ringer's solution, lactate-Ringer's solution, or any isotonic physiologically acceptable solution for administration by the chosen means. Preferably, the solution is sterile and pyrogen-free, and is manufactured and packaged under current Good Manufacturing Processes (GMPs), as approved by the FDA. The clinician of ordinary skill is familiar with appropriate ranges for pH, tonicity, and additives or preservatives when formulating pharmaceutical compositions for administration by intravascular injection, intrathecal injection, injection into the cerebro-spinal fluid, direct injection into the tumor, or by other routes.

In addition to additives for adjusting pH or tonicity, the antibody-therapeutics and antibody-imaging agents may be stabilized against aggregation and polymerization with amino acids and non-ionic detergents, polysorbate, and polyethylene glycol. Optionally, additional stabilizers may include various physiologically-acceptable carbohydrates and salts. Also, polyvinylpyrrolidone may be added in addition to the amino acid. Suitable therapeutic immunoglobulin solutions which are stabilized for storage and administration to humans are described in U.S. Pat. No. 5,945,098, incorporated fully herein by reference. Other agents, such as human serum albumin (HSA), may be added to the therapeutic or imaging composition to stabilize the antibody conjugates.

The compositions of the invention may be administered using any medically appropriate procedure, e.g., intravascular (intravenous, intraarterial, intracapillary) administration, injection into the cerebrospinal fluid, intracavity or direct injection in the tumor. Intrathecal administration maybe carried out through the use of an Ommaya reservoir, in accordance with known techniques. (F. Balis et al., Am J. Pediatr. Hematol. Oncol. 11, 74, 76 (1989). For the imaging compositions of the invention, administration via intravascular injection is preferred for pre-operative visualization of the tumor. Post-operative visualization or visualization concurrent with an operation may be through intrathecal or intracavity administration, as through an Ommaya reservoir, or also by intravascular administration.

Intravascular injection may be by intravenous or intraarterial injection. Antibody-agents injected into the blood stream have been shown to cross the blood-brain barrier and to infiltrate the cranial cavity to some extent, usually in the range of 10⁻⁴ to 10⁻³% injected dose per gram. This rate of uptake may be sufficient for imaging reagents, and also may be useful for tumor cell specific cytotoxic agents (e.g, those specifically directed to the inhibition of the function of tumor-cell overexpressed proteins). However, in order to achieve therapeutic concentrations of the antibody-therapeutic agents without unacceptable toxicity to the patient, it is preferred that the therapeutics compositions be administered by intrathecal injection, direct injection, or injection into the cerebro-spinal fluid.

A preferred method for administration of the therapeutic compositions of the invention is by depositing it into the inner cavity of a cystic tumor by any suitable technique, such as by direct injection (aided by stereotaxic positioning of an injection syringe, if necessary) or by placing the tip of an Ommaya reservoir into a cavity, or cyst, for administration. Where the tumor is a solid tumor, the antibody may be administered by first creating a resection cavity in the location of the tumor. This procedure differs from an ordinary craniotomy and tumor resection only in a few minor respects. As tumor resection is a common treatment procedure, and is often indicated to relieve pressure, administration of the therapeutic compositions of the invention following tumor resection is a preferred embodiment of the treatment methods of the invention. Following gross total resection in a standard neurosurgical fashion, the cavity is preferable rinsed with saline until all bleeding is stopped by cauterization. Next the pia-arachnoid membrane, surrounding the tumor cavity at the surface, is cauterized to enhance the formation of fibroblastic reaction and scarring in the pia-arachnoid area. The result is the formation of an enclosed, fluid-filled cavity within the brain tissue at the location from where the tumor was removed. After the cyst has been formed, either the tip of an Ommaya reservoir or a micro catheter, which is connected to a pump device and allows the continuous infusion of an antibody solution into the cavity, can be placed into the cavity. See, e.g., U.S. Pat. No. 5,558,852, incorporated fully herein by reference.

Alternatively, a convection-enhanced delivery catheter may be implanted directly into the tumor mass, into a natural or surgically created cyst, or into the normal tissue mass. Such convection-enhanced pharmaceutical composition delivery devices greatly improve the diffusion of the composition throughout the tissue mass. The implanted catheters of these delivery devices utilize high-flow microinfusion (with flow rates in the range of about 0.5 to 15.0 μl/minute), rather than diffusive flow, to deliver the therapeutic or imaging composition to the brain and/or tumor mass. Such devices are described in U.S. Pat. No. 5,720,720, incorporated fully herein by reference.

The effective amount of the therapeutic antibody-conjugate composition or of the imaging antibody-conjugate compositions to be given to a particular patient will depend on a variety of factors, several of which will be different from patient to patient. A competent clinician will be able to determine an effective amount of a therapeutic antibody-conjugate composition to administer to a patient to retard the growth and promote the death of tumor cells, or an effective amount of an imaging composition to administer to a patient to facilitate the visualization of a tumor. Dosage of the antibody-conjugate will depend on the treatment of the tumor, route of administration, the nature of the therapeutics, sensitivity of the tumor to the therapeutics, etc. Utilizing LD₅₀ animal data, and other information available for the conjugated cytotoxic or imaging moiety, a clinician can determine the maximum safe dose for an individual, depending on the route of administration.

For instance, an intravenously administered dose may be larger than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Imaging moieties are typically less toxic than cytotoxic moieties and may be administered in higher doses in some embodiments. Utilizing ordinary skill, the competent clinician will be able to optimize the dosage of a particular therapeutic or imaging composition in the course of routine clinical trials.

Typically the dosage will be 0.001 to 100 milligrams of conjugate per kilogram subject body weight. Doses in the range of 0.01 to 1 mg per kilogram of patient body weight may be utilized for a radionuclide therapeutic composition that is administered intrathecally. Relatively large doses, in the range of 0.1 to 10 mg per kilogram of patient body weight, may used for imaging conjugates with a relatively non-toxic imaging moiety. The amount utilized will depend on the sensitivity of the imaging method, and the relative toxicity of the imaging moiety. In a therapeutic example, where the therapeutic composition comprises a ¹³¹I cytotoxic moiety, the dosage to the patient will typically start at a lower range of 10 mCi, and go up to 100, 300 or even 500 mCi. Stated otherwise, where the therapeutic agent is ¹³¹I, the dosage to the patient will typically be from 5,000 Rads to 100,000 Rads (preferably at least 13,000 Rads, or even at least 50,000 Rads). Doses for other radionuclides are typically selected so that the tumoricidal dose will be equivalent to the foregoing range for ¹³¹I. Similarly, chemotoxic or toxin protein doses may be scaled accordingly.

The antibody conjugate can be administered to the subject in a series of more than one administration. For therapeutic compositions, regular periodic administration (e.g., every 2-3 days) will sometimes be required, or may be desirable to reduce toxicity. For therapeutic compositions which will be utilized in repeated-dose regimens, antibody moieties which do not provoke HAMA or other immune responses are preferred. The imaging antibody conjugate compositions may be administered at an appropriate time before the visualization technique. For example, administration within an hour before direct visual inspection may be appropriate, or administration within twelve hours before an MRI scan may be appropriate. Care should be taken, however, to not allow too much time to pass between administration and visualization, as the imaging compound may eventually be cleared from the patient's system.

In addition to the use of imaging antibody conjugates for simple visualization, these compositions may be utilized as a “dry run” for more toxic cytotoxic antibody conjugates. If the same antibody moiety is utilized for the imaging conjugate as for the therapeutic conjugate, the physician may first use a visualization technique to determine precisely where in the brain the cytotoxic conjugate will concentrate. If a sufficient degree of tissue selectivity is not achieved (e.g, if the tumor cells are too disperse in the normal tissue, or if the particular brain tumor protein target chosen is not sufficiently overexpressed in the particular patient's tumor cells), then the physician may choose another brain tumor protein target. The provision of numerous brain tumor protein targets by the present invention, along with both imaging and therapeutic agents, allows a high degree of flexibility in designing an effective treatment regimen for the individual patient.

### Combination Therapies of the Invention

As mentioned previously, many tumors are heterogeneous in character, and pervasive throughout tissue. This combination often makes them difficult to treat, as individual portions of the tumor cells in any particular patient may have differing biological characteristic. Thus, in some cases, it may be preferred to use various combinations of therapeutic or imaging agents, in order to more fully target all of the cells exhibiting tumorigenic characteristics. Such combination treatments may be by administering blended antibody therapeutic or imaging compositions, individually prepared as described above, and administering the blended therapeutic to the patient as described. The skilled administering physician will be able to take such factors as combined toxicity, and individual antibody agent efficacy, into account when administering such combined agents. Additionally, those of skill in the art will be able to screen the antibodies to avoid potential cross-reaction with each other, in order to assure full efficacy of each antibody therapeutic or imaging agent.

Alternatively, several individual tumor-targeted compositions may be administered simultaneously or in succession for a combined therapy. This may be desirable to avoid accumulated toxicity from several antibody conjugate reagents, or to more closely monitor potential adverse reactions to the individual antibody reagents. Thus, cycles such as where a first antibody therapeutic agent is administered on day one, followed by a second on day two, then a period with out administration, followed by re-administration of the antibody therapeutics on different successive days, is comprehended within the present invention.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### EXAMPLE 1

### Identification of Two New Splicing Variant Isoforms of PTPζ: PTPζ SM1 and SM2

The mRNA nucleotide sequence for PTPζ SM1 was identified in a human fetal brain phage cDNA library by sequencing.

The mRNA nucleotide sequence for PTPζ SM2 was identified by PCR amplification of adult human brain cDNA, and sequencing of the resulting nucleic acids.

For the RT-PCR analyses performed below, total RNA was isolated from either cells (glioblastoma cultured lines) or tissue using Trizole (Gibco Life Technologies, Inc.), following the manufacture's protocol. cDNA was generated from total RNA using the 1^{st} Strand synthesis kit from Gibco Life Technologies, Inc., and an oligo dT₃₀ anchored primer. For each RT-PCR reaction, 1 μl of cDNA was utilized. The PCR reaction was carried out using an Advantage 2 kit (Clontech) under standard conditions. The products of the PCR reactions were confirmed via sequencing.

Both clones were verified by RT-PCR analysis of glioblastoma cell lines and primary tumors. For PTPζ SM1, primers CAGCAGTTGGATGGAAGAGGAC [SEQ ID NO. 7] and CACTGAGATTCTGGCACTATTC [SEQ ID NO. 8] were used, producing an identifiable 1116 bp product. RT-PCR analysis was performed, confirming expression of the SM1 splice variant in 11 of 17 different glioblastoma cell lines tested, fetal brain, and adult brain, using the unique 3′ end and portion of the 3′ untranslated region as the hybridization target for the probe. In addition, RT-PCR analysis was performed on 28 primary brain tumor samples, confirming expression of the PTPζ SM1 variant in 16 of the 28 tumors.

For PTPζ SM2, primers AACMTTCCAGGGTCTCACTC [SEQ ID NO. 9] and TTGACTGGCTCAGGAGTATAG [SEQ ID NO. 10] were used, which produce a 130 bp product when the extra exon 23a is present, and a no product when the exon 23a is absent. RT-PCR analysis was performed, confirming expression in 6 of 17 different glioblastoma cell lines tested. In addition, RT-PCR analysis was performed on 28 primary brain tumor samples, confirming expression of the PTPζ SM1 variant in 19 of the 28 tumors.

For comparison, RT-PCR analysis was also done for the expression of PTPζ-α (primers CTGATAATGAGGGCTCCCAAC [SEQ ID NO. 11] and CTCTGCACTTCCTGGTAAAACTCT [SEQ ID NO. 12]) and PTPζ-β (primers CAGCAGTTGGATGGAAGAGGAC [SEQ ID NO. 13] and CTCTGCACTTCCTGGTAAAACTCT [SEQ ID NO. 14]) in the 28 brain tumor tissue samples. PTPζ-α was shown to be expressed in 16 of the 28 samples, and the short form PTPζ-β was shown to be expressed in 19 of the 28 samples.

The nucleotide sequence alignment of the two new splice variants with the reference sequence for PTPζ-α is shown in the following table:

TABLE 1

| PTP 5′ | PTP 3′ | PAC 1 5′ | PAC 1 3′ | Corresponding Exon Key: | |
|---|---|---|---|---|---|
| 1 | 48 | 87274 | 87321 | 5′ UTR PAC 1: RF5-1062J16 | |
| | | | | | BAC: RP11-384A20 |
| 70 | 205 | 87343 | 87487 | 1 | PAC 2: RP5-1049N15 |
| 205 | 272 | 142076 | 142143 | 2 | |
| | | BAC 5′ | BAC 3′ | | |
| 291 | 451 | 24001 | 24161 * | 3 | * 88 nt deletion seen in 5′ PCR clone from PTP 363-451 |
| 450 | 603 | 28570 | 28723 | 4 | |
| 602 | 701 | 32814 | 32888 | 5 | |
| 698 | 772 | 32814 | 32888 | 6 | |
| 766 | 924 | 39695 | 39853 | 7 | |
| 922 | 1075 | 39995 | 40148 | 8 | |
| 1074 | 1261 | 52411 | 52598 * | 9 | * not spliced at 1261 in phage library clones |
| 1260 | 1387 | 53910 | 54037 | 10 | |
| 1387 | 1435 | 60644 | 60692 | 11 | |

| 1432 | 2346 | 66362 | 67276 | 5′ 12 (end of BAC) |
|---|---|---|---|---|
| PAC 2 5′ | | PAC 2 3′ | | |
| 2147 | 4409 | 1 | 2263 | mid 12 |
| 4437 | 4987 | 2294 | 2844 | 3′ 12 |
| 4925 | 5133 | 8027 | 8224 | 13 |
| 5131 | 5224 | 17505 | 17598 | 14 |
| 5223 | 5310 | 20427 | 20514 | 15 |
| 5309 | 5332 | 23048 | 23071 | 16 |
| 5329 | 5428 | 23234 | 23333 | 17 |
| 5429 | 5512 | 25555 | 25638 | 18 |
| 5512 | 5646 | 27710 | 27844 | 19 |
| 5572 | 5602 | 42925 | 42955 * | Duplicate of mid 19 * duplicated regions of exons 19 |
| 5646 | 5768 | 28408 | 28530 | most of 20 (−12 bp 3′) and 26 vary by one aa / |

two nt

| 5791 | 5945 | 29770 | 29934 | 21 (−10 bp 5′) | |
|---|---|---|---|---|---|
| 5943 | 6082 | 31560 | 31699 | 22 | |
| 6080 | 6228 | 33375 | 33523 ˜ | 23 | ˜116 nt insert seen b/w exons 23 & 24 |

in 3′ PCR clone: maps to PAC b/w 23 & 24

| 6225 | 6322 | 40379 | 40476 ˜ | 24 | PTP location PAC 2 5′ PAC 2 3′ | | |
|---|---|---|---|---|---|---|---|
| 6322 | 6397 | 40820 | 40895 | 25 | 6228 | 36744 | 36629 |

| | | | | |
|---|---|---|---|---|
| 6396 | 6526 | 42864 | 42994 | 26 |
| 6457 | 6487 | 27770 | 27800 * | Duplicate of mid 26 |
| 6525 | 6673 | 43895 | 44043 | 27 |
| 6671 | 6816 | 47753 | 47898 | 28 |
| 6816 | 6952 | 48708 | 48844 | 29 |

**BOUNDARIES DETERMINED FROM HOMO SAPIENS CHROMOSOME 7 WORKING DRAFT (NT_007845.3)
**Nucleotide location refers to position in full length RPTPZ (accession M93426)

### EXAMPLE 2

### Cell Migration Assay For Determining Antibody Activity on Protein Targets

Tumor cells are known to migrate more rapidly towards chemoattractants. The cell migration assay measures the ability of a cell to migrate. The ability to migrate is taken as a measure of tumorigenicity. Chemoattractants generally used are fetal bovine serum, pleiotrophin, bFGF, and VEGF. Thus, this assay can be used to determine migration capability of a cell in which the gene has been knocked down or the gene of interest is being overexpressed.

The ChemoTx® disposable chemotaxis system (Neuroprobe, Inc., Gaithersburg, Md.) is used according to the manufacturer's instructions, with a few modifications. Briefly, gliobastoma cultured cells from cell line G55T2 are prepared by splitting the cells the day before the assay is performed. A ChemoTx® chamber with the following specifications is used: Pore size 8 μm, exposed filter area 8 mm², exposed filter area diameter 3.2 mm. The plate configuration is: 30 μl per well, 96 well plate. The membrane type is: Track-etched polycarbonate.

In preparation for the assays, the filter membrane is coated in 100 ml PBS containing 0.1% acetic acid and 3.5 ml Vitrogen 100 (from Cohesion) at 37° C. overnight. About 30 minutes before starting the assay the coated membrane is washed and rinsed with PBS containing 0.1% BSA. Cells are harvested by using the standard technique (trypsin-EDTA). The cells are washed once with DMEM 10% FBS, and then spun at 1000 RPM, for 5 minutes at room temperature. The pellet is resuspended in DMEM without serum, containing 0.1% BSA (serum free medium). The cells are spun and resuspended again in serum free medium, and then spun and resuspended in the amount of serum free medium needed to provide a concentration of 1 mio. cells/ml, or 25,000 cells per 25 ul. Just prior to the assay, a suitable amount of the antibody to be tested for anti-target function activity is added to the cell suspension.

For the assay, a standard chemoattractant is used to measure the mobility of the cells. The chemoattractants are diluted in serum free medium. A suitable unspecific chemoattractant is DMEM with 5% FBS. The chemoattractant solutions and control solutions without chemoattractant are pipetted (29 μl) into the lower plate wells. After placing and securing the filter plate over the lower wells, ensuring contact with the solution in the bottom wells, serial dilutions of the cell suspension are pipetted onto each site on the filter top. The plates are them covered and incubated at 37° C., 5% CO₂, for 3-4 hours.

After incubation, the upper filter side is rinsed with PBS and exposed upper filter areas are cleaned with wet cotton swabs. The filter is stained using Diff-Quik™ (VWR) dye kit, according to the manufacturer's instructions. The migrated cells are counted on the lower filter side using a microscope (Magnification 200×), by counting of 5 high power field sections per well.

### EXAMPLE 3

### HUVEC (Human Umbilical Vein Endothelial Cells) Endothelial Sprouting Assay for Determining Antibody Activity on Protein Targets

Cell-sprouting morphology are utilized as an easily visualized assay to determine the inhibitory effect of a candidate antibody on the protein target function for protein targets which stimulate endothelial cell sprouting, such as PTPζ. Such assays have been described extensively in the literature (Nehls, V., et al., Histochem. Cell Biol. 104: 459-466 (1995); Koblizek, T. I., et al., Curr. Biol. 8: 529-532 (1988); and Kwak, H. J., et al., FEBS Lett. 448: 249-253). Briefly, a endothelial cells from a suitable source, such as HUVECs or PPAECs (porcine pulmonary artery endothelial cells) are grown to confluence on microcarrier (MC) beads (diameter 175 μm, available from Sigma) and placed into a 2.5 mg/ml fibrinogen gel containing the protein target at an appropriate effective concentration (200 ng/ml is an suitable starting concentration, which the skilled practitioner may optimize) and the antibody in an appropriate range of concentrations (this will depend on antibody titer and affinity for the target), and 200 units/ml Trasylol (available from Bayer). Fibrin gels are incubated in M-199 with a daily supplement of the same amount of recombinant protein and antibody, 2.0% heat-inactivated fetal bovine serum, and 200 units/ml Trasylol. After three days, the extent of sprouting is determined using a phase-contrast microscope (such as those available from Zeiss). A decrease in cell sprouting as compared to controls without antibody indicates a reduction in protein target activity by the antibody.

### EXAMPLE 4

### Antibody Production and Affinity

Custom mouse monoclonal antibodies were generated to the extracellular domain of recombinant human PTPζ-β form. The extracellular domain for PTPζ-β (residues 26-774), including the unique splice junction, was expressed with a C-terminus 6× His tag using baculovirus. The protein was purified from the media with two chromatography steps: immobilized metal affinity (Ni²⁺-NTA FF, Qiagen) followed by anion exchange (Q Sepharose FF, Amersham Biosciences) (Lorente et al., 2005). Mouse hybridomas were generated by Anaspec using BALB/c mice with the protein immunogen in Freund's adjuvant. Spleen cells from immunized mice were fused with a mouse myeloma cell line. Supernatants were screened by ELISA using the PTPζ protein immunogen, isotyped and PTPζ specific IgG producing hybridomas expanded, subcloned and cultured. Promising hybridoma lines were grown and the antibodies purified from culure media using Protein A/G Montage Purification columns (Millipore).

Antibodies to PTPζ were screened by ELISA to determine specificity. To determine the specificity of antibody containing hybridoma supernatants and purified antibody preparations, 96 well Maxisorp plates (Nunc) were coated in 0.5 mM sodium bicarbonate solution containing 1 μg/ml antigen for 1 hr at room temperature. The plates were then washed with PBS and blocked with BSA for 1 hr. Antibodies were added for 1 hr and then washed three times with PBS containing 0.1% TWEEN-20 to remove unbound or non-specific proteins. The anti-mouse IgG-horse radish peroxidase detection antibody was then added for 1 hr prior to the final washes in PBS containing 0.1% TWEEN-20. The immune complex was incubated briefly with TMB substrate (Sigma), stopped with 0.1 N HCl and absorbance detected at 490 nm on a plate reader.

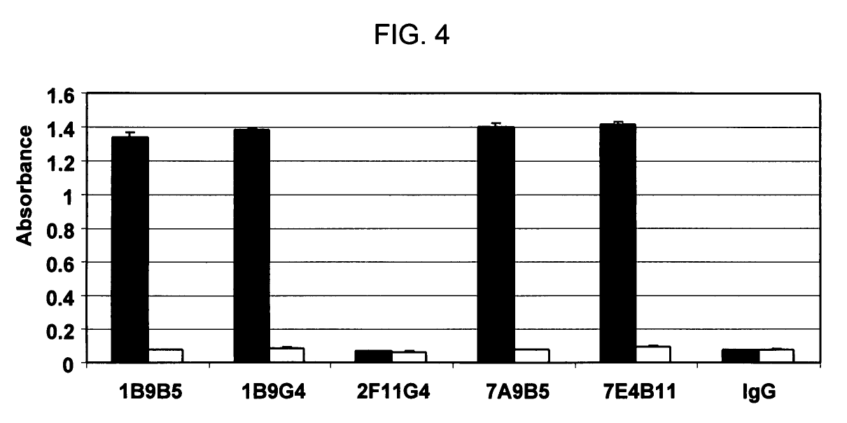

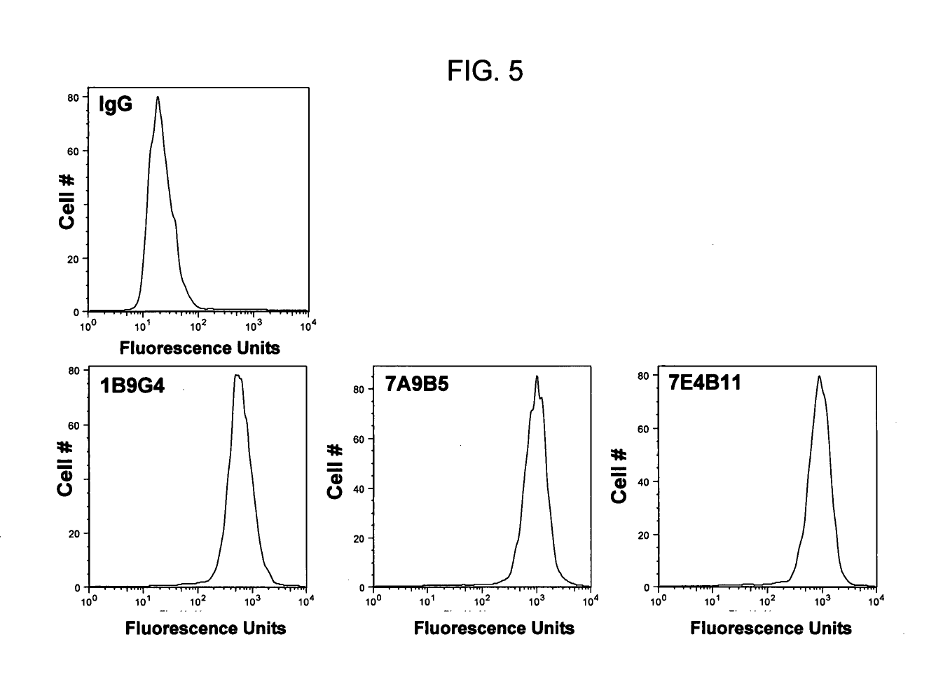

In the ELISA experiment, a panel of purified antibodies was tested for binding to recombinant PTPζ-β extracellular domain protein antigen (PTPζ-β-ECD-black bars) or a unrelated similarly expressed and purified control antigen, (NR-ECD-grey bars). In this example four of the five PTPζ antibodies tested gave a robust and specific signal for PTPζ-β-ECD and did not recognize the non-related protein (FIG. 4). The hybridomas corresponding to three of the four positive antibodies were selected and used for further study.

Affinity Measurements—Surface Plasmon Resonance.

Biacore utilizes a sensor chip technology for monitoring interaction between two or more molecules in real time, without the use of labels. The antibodies were captured on the sensor via anti-mouse IgG1 pre-immobilized on the chip surface. The running buffer was HBS-EP (50 mM HEPES, pH 7.4, 150 mM NaCl, 3 mM EDTA, and 0.005% Surfactant P-20) and the analysis temperature was 25° C. The recombinant human PTPζ-β was injected, using an automated method, up to 10 minutes at flow rates ranging from 10-50 μl/min. Binding data was fit to a 1:1 binding model using Biacore software (BIAevaluation v 4.1) to obtain the kinetic and affinity constants (Table 2). Three antibodies had similar low nM affintities (˜K_{D}8 nM).

TABLE 2
Affinity of PTPζ antibodies

| Anti- PTPζ Ab | kₐ (M⁻¹s⁻¹) | k_{d} (s⁻¹) | K_{D} (nM) |
|---|---|---|---|
| 1B9G4 | 8.2 × 10⁴ | 6.7 × 10⁻⁴ | 8.2 |
| 7A9B5 | 7.6 × 10⁴ | 6.4 × 10⁻⁴ | 8.5 |
| 7E4B11 | 6.9 × 10⁴ | 6.5 × 10⁻⁴ | 9.4 |

The antibodies were compared to prior art and commercially available antibodies. A summary of the comparison is shown in Table 3.

TABLE 3

| Antibody | Host & | | | Species | |
|---|---|---|---|---|---|
| Name | Isotype | Source | Antigen | Specificity | Utility |
| 7E4B11 | Mouse | AGY | RPTPβ | Human | Human Diagnostic and |
| | IgG1 | Therapeutics, Inc. | extracellular | | Therapeutic |
| | | | domain | | |
| Anti- | Mouse | Transduction | RPTPβ | Human | Immunoblot, |
| RPTPβ | IgG1 | Labs (Becton, | intracellular | Reactivity | Immunofluorescence |
| | | Dickinson & Co.) | domain | to rat, | Immunohistochemistry |
| | | | | mouse | for intracellular antigen |
| 2B49 | Mouse | Margolis et al.* | RPTPβ | Rat | Immunoblot (rat |
| | IgG1 | | extracellular | | lysates) |
| | | | domain | | |
| 3F8 | Mouse | Margolis et al. | Rat RPTPβ | Rat | Immunoblot (rat |
| | IgG1 | | extracellular | | lysates) |
| | | | domain | | |
| MAB5210 | Mouse | Chemicon | phosphacan | Human | Immunoblot (rat |
| | IgM | International | domain | Reactivity | lysates), |
| | | | (extracellular) | to rat, | Immunocytochemistry |
| | | | | other | Immunoprecipitation |
| | | | | species | Immunohistochemistry |
| | | | | not tested | |

*J. Biol. Chem. 266, 14785-14801; Proc. Natl. Acad. Sci 91:2512-2516.

Previously known antibodies suffer from disadvantages for human diagnostics or therapeutic applications. The prior art antibodies that recognize human RPTP react with intracellular domains, or are IgM isotype, which has a high non-specific signal and limitations with detection methods. Consequently they are not used for therapeutics. Other published anti-RPTPβ antibodies are not suitable for human diagnostics or therapeutic application because they do not recognize human RPTPβ.

### EXAMPLE 5

### PTPζ Expression Studies

A survey of several tumor tissues revealed that PTPζ is overexpressed in many tumor types. Tissue MicroArray slides were used to study the expression PTPζ. Slides were placed on a heat block at 45° C. for 4-6 hrs and dewaxed using EZ-DeWax solution (Innogenex). Slides were then placed in a bath containing Target Retrival Solution (Innogenex) and simmered for 15 minutes in a microwave. Slides were stained with either the carboxy terminus PTPζ antibody (Transduction Labs) or the custom made PTPζ mouse monoclonal antibodies (Anaspec). The slides were processed using either anti-mouse or anti-rabbit immunohistochemistry kits with DAB calorimetric end-point detection and hemotoxylin counterstain (Innogenex).

Immunohistochemistry analysis of normal and tumor tissue with PTPζ-7E4B11 antibody. A panel of normal human tissue was surveyed for expression of PTPζ using the 7E4B11 antibody. None of the normal peripheral tissues examined displayed significant staining with 7E4B11; adrenal, uterus, lung, pancreas, testicle, kidney, spleen, thyroid, lymph node, and liver. A glioblastoma tumor specimen that shows positive staining with 7E4B11 is included in the study as a reference. A panel of human tumor tissue was surveyed for expression of PTPζ antibody. Normal colon had modest expression in some duct cells, while colon adenocarcinoma was positive for PTPζ. Normal breast had low expression in some duct cells, while invasive ductal carcinoma of the breast stained intensely for PTPζ. Normal lung did not stain for PTPζ, but lung adenocarcinoma displayed strong PTPζ immunoreactivity. Normal skin did not stain with PTPζ, but melanoma was immunoreactive.

TABLE 4
PTPζ expression in tumor tissue

| Cancer Tissue | Histology | Positive Tumors | |
|---|---|---|---|
| Breast | Adenocarcinoma | 6 of 15 | (40%) |
| Ovary | Cystadenocarcinoma | 6 of 10 | (60%) |
| Endometrium | Adenocarcinoma | 2 of 7 | (28%) |
| Stomach | Adenocarcinoma | 4 of 5 | (80%) |
| Colon | Adenocarcinoma | 10 of 11 | (91%) |
| Pancreas | Adenocarcinoma | 0 of 9 | (0%) |
| Liver | Hepatocarcinoma | 3 of 6 | (50%) |
| Renal/Pelvis | Transitional Carcinoma | 4 of 8 | (50%) |
| Kidney | Renal Carcinoma | 8 of 15 | (53%) |
| Bladder | Transitional Carcinoma | 14 of 20 | (70%) |
| Prostate | Adenocarcinoma | 6 of 14 | (43%) |
| Skin | Melanoma | 3 of 5 | (60%) |
| Esophagous | Adenocarcinoma | 5 of 6 | (83%) |
| Lip/Tongue/ | Squamous | 23 of 28 | (82%) |
| Mouth | | | |
| Paratoid | Mixed Tumor | 4 of 7 | (57%) |
| Larynx | Squamous | 5 of 5 | (100%) |
| Pharynx | Squamous | 6 of 6 | (100%) |
| Lymph Node | Lymphoma | 4 of 7 | (57%) |
| Lung | Squamous/Adeno. | 2 of 10 | (20%) |

### EXAMPLE 6

### Colony Formation in Soft Agar

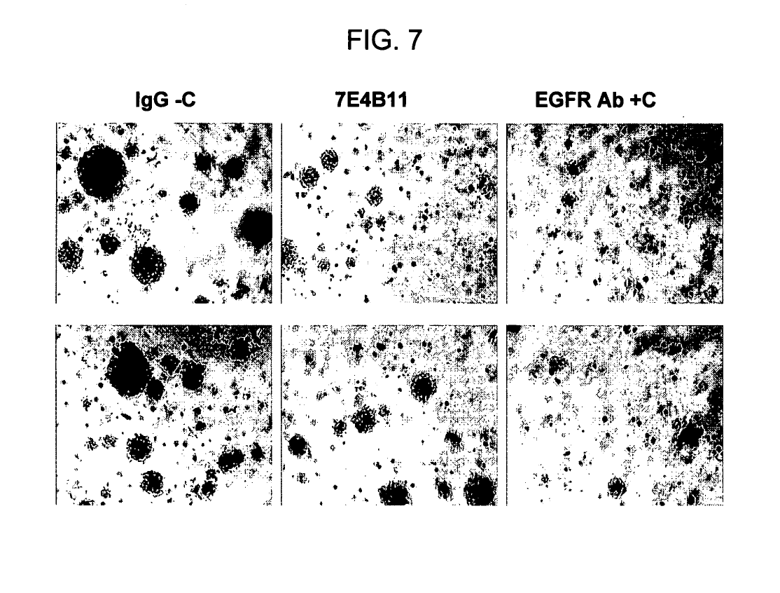

We then set out to test if 7E4B11 could inhibit or prevent colony formation in soft agar. To test the effect of 7E4B11 on colony formation a cell transformation detection assay kit (Chemicon) was used. Briefly, 24 well plates were prepared with 0.8% base agar in cell culture media (DMEM, 10% FBS, 1% Penicillin/Streptomycin). U87 cells were harvested and mixed at 4000 cells/well in 0.4% top agar solution in cell culture media. Media containing IgG1 (Cymbus Biotechnology) 20 μg/ml, EGFR-528 (Santa Cruz Biotechnology) 20 μg/ml, or 7E4B11 20 μg/ml was added in triplicate to the appropriate wells. The cells were incubated for 21 days at 37° C. in 5% CO2 with media treatment changes every 3-4 days. At that time colonies were stained and visualized using light microscopy. (FIG. 7). Control IgG1 antibody did not inhibit colony formation in soft agar. In contrast, the positive control EGFR antibody and 7E4B11 had fewer and smaller colonies. Therefore, 7E4B11 is able to modulate PTPζ function and thereby inhibit U87 tumor formation in soft agar.

### EXAMPLE 7

### PTPζ Immunotoxin Mediated Cell Cytotoxicity

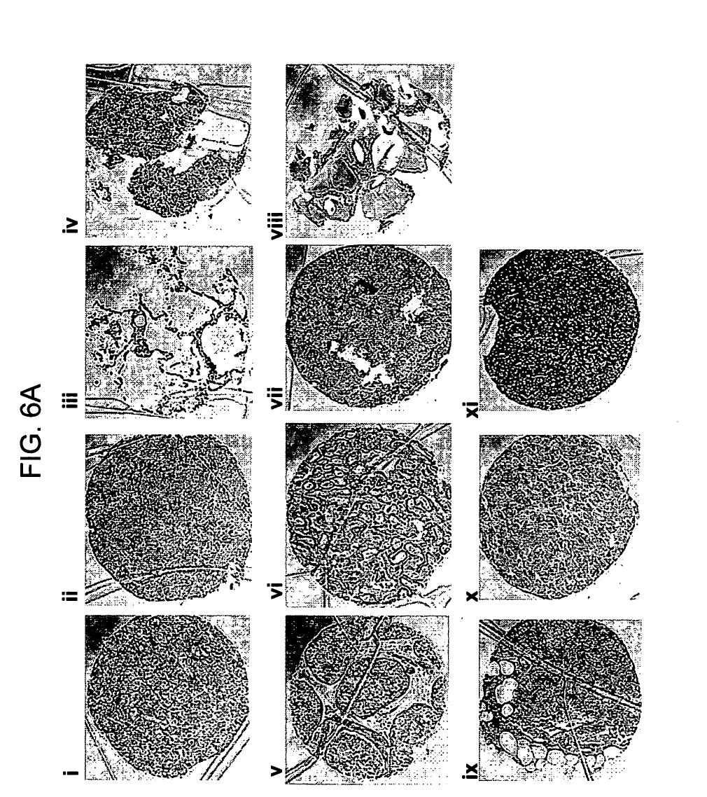

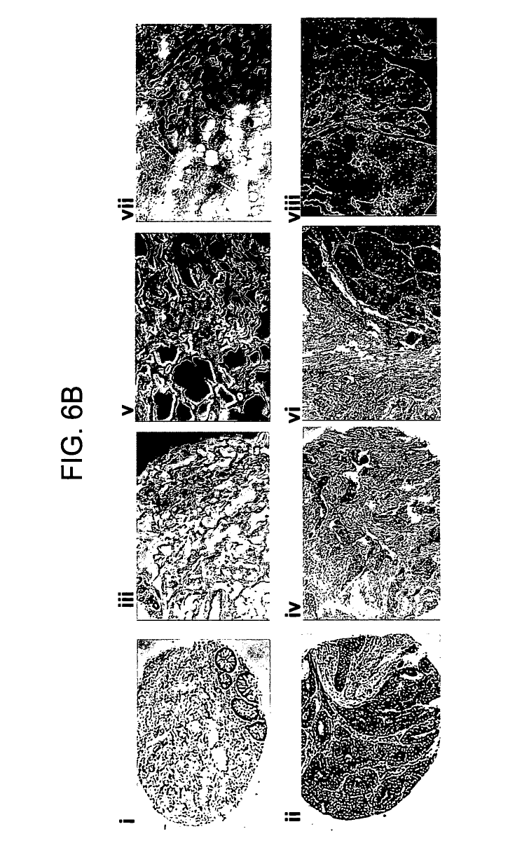

To evaluate if the PTPζ mouse antibodies can act as an immunotoxin we tested indirect and direct immunotoxin mediated cell cytotoxicity assays. For indirect immunotoxin assay, U87 cells were plated at 3,000 cells/well (30,000 cells/ml) onto white walled 96 well plates (BD-Falcon) and incubated overnight at 37° C. in 5% CO2. The cells were treated with 200 ng/well primary antibody prepared in Optimem (Gibco). The antibody treatments include the PTPζ antibodies as well as EGFR-528 as positive control (Santa Cruz Biotechnology), CD71 as positive control (Transduction Labs), and IgG1 (Cymbus Biotechnology). The negative controls included an isotype control Ab and media alone. Half of the test wells were subsequently treated with Saporin conjugated secondary antibody (MAB-ZAP) at 100 ng/well. The cells were then incubated for 3 days 37° C. in 5% CO₂. If the primary antibody recognizes its target and gets internalized, then the toxin-antibody complex is delivered and kills the cells. The number of viable cells were assessed using the luminescence based detection reagent Cell Titer Glo (Promega) and read on a luminometer. FIG. 6A demonstrates that the PTPζ Abs (1B9G4, 7A9B5, and 7E4B11), as well as the positive control Abs, effectively kill tumor cells when coupled to the anti-mouse IgG immunotoxin. The isotype control antibody was unable to bind and internalize and thus could not kill these cells.

Purified RPTPβ-7E4B11 and -7A9B5 antibodies were directly conjugated to Saporin (Advanced Targeting Systems) and evaluated in cell culture for the ability to kill glioma cells. In this experiment, glioma cells are treated with these PTPζ immunotoxins as well as control immunotoxins. The negative controls included an isotype control Ab (IgG Neg. Ctrl) or media alone (Vehicle). The positive control Ab (DAT-SAP) targets the Dopamine Transporter expressed on astrocytoma cells (data not shown). If the immunotoxin recognizes its target and gets internalized, then the toxin payload is delivered and kills the cells. FIG. 6B demonstrates that the immunotoxins 7A9B5-SAP and 7E4B11-SAP, as well as the positive control Ab, effectively kill tumor cells when directly coupled to the toxin. The isotype control antibody were unable to bind and internalize and thus could not kill cells.

### EXAMPLE 8

### Tumor Xenografts

Female athymic nude mice were 11-12 weeks old on day 1 of the study. The U87MG glioblastoma line used for this study was maintained in athymic nude mice. A tumor fragment (1 mm³) was implanted s.c. into the right flank of each test mouse. Tumors were monitored twice weekly and then daily as their volumes approached 120-160 mm³ at which time the treatment began. IgG-saporin, 7E4B11-saporin doses were prepared on each day of dosing by dilution with saline. The Ab treated groups received 15 and 30 μg/mouse, 2×/wk×3 i.t. Each animal was euthanized when its neoplasm reached the predetermined endpoint size (1,500 mm³). The logrank test was employed to analyze the significant differences of time to endpoint (TTE).

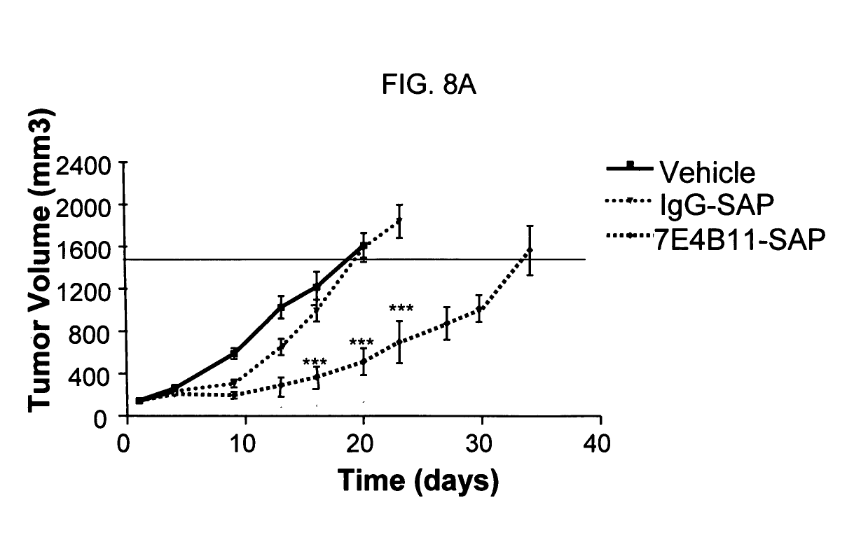

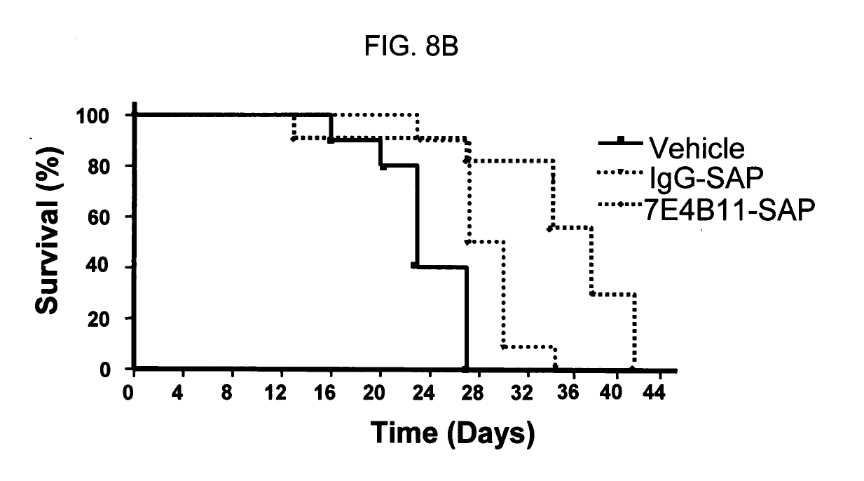

Non-targeted IgG1-SAP treatments at 15 ug/dose produced only 2% tumor growth delay (TGD). However, at 30 μg/dose, IgG1-SAP produced 24% TGD. At 15 and 30 μg/dose, 7E4B11-SAP produced 25% and 73% TGD and were highly significant (P<0.001) (FIG. 8A). All tumors reached the endpoint volume by day 44. The median TTE of PBS-treated mice was 18.6 days. Whereas the 7E4B11-SAP (30 μg/dose) treated mice had a TTE of 32.1 days (FIG. 8B). These results demonstrate that 7E4B11-SAP has significant antitumor activity.

We then set out to determine the antitumor activity of unconjugated 7E4B11 in the human U87MG glioblastoma xenograft model. Nude mice received intraperitoneal injections of 7E4B11, control IgG or PBS twice weekly for two weeks. All treatments began on day 1 in groups of 10 mice bearing well established (130 mm³) U87MG glioblastomas. Intraperitoneal 7E4B11 (20 μg/dose) treatment produced 27% tumor growth delay (P<0.05) relative to the vehicle, while the IgG treatment group did not demonstrate statistically significant anti-tumor activity. In addition, 7E4B11 treatment yielded two 60-day survivors with partial regression responses. The 7E4B11 unconjugated antibody was well tolerated and no toxic deaths were recorded.

1. A method to treat a tumor comprising:
administering a therapeutic amount of a composition comprising a compound of the general formula α(PTPζ), wherein α(PTPζ) specifically binds human protein tyrosine phosphatase-zeta and a pharmaceutically acceptable carrier, wherein said tumor is selected from the group consisting of invasive ductal carcinoma of the breast; colon adenocarcinoma; transitional carcinoma of the bladder; and squamous cell carcinoma of the oral cavity and pharanx;
wherein said composition inhibits cell growth or promotes cell death of said tumor.

2. The method of claim 1 wherein the therapeutic composition is administered by intrathecal administration.

3. The method of claim 1 wherein the therapeutic composition is administered by intravascular administration.

4. The method of claim 1 wherein α(PTPζ) is selected from the group consisting of an antibody and an antibody fragment.

5. The method of claim 4 wherein α(PTPζ) is an antibody selected from the group consisting of: monoclonal antibodies, polyclonal antibodies, humanized antibodies, recombinant antibodies, chemically modified antibodies, and synthetic antibodies.

6. The method of claim 4 wherein α(PTPζ) is an antibody fragment selected from the group consisting of fragments of: monoclonal antibodies, polyclonal antibodies, humanized antibodies, recombinant antibodies, chemically modified antibodies, and synthetic antibodies.

7. The method of claim 1 wherein α(PTPζ) comprises a cytotoxic moiety.

8. The method of claim 7 wherein the cytotoxic moiety comprises a pharmaceutically acceptable radioactive isotope.

9. The method of claim 7 wherein the cytotoxic moiety is chemotoxic.

10. The method of claim 7 wherein the cytotoxic moiety is a toxin protein.

11. The method of claim 1, wherein said antibody specifically binds to the extracellular domain of PTPζ-β.

12. A method to treat a brain tumor comprising administering a therapeutic amount of a composition comprising:
a compound of the general formula α(PTPζ), wherein α(Pζ) specifically binds the extracellular domain of human protein tyrosine phosphatase-zetaand a pharmaceutically acceptable carrier.

13. The method of claim 12 wherein the brain tumor is a glioblastoma.

14. The method of claim 12 wherein α(PTPζ) is selected from the group consisting of an antibody and an antibody fragment.

15. The method of claim 14 wherein α(PTPζ) is an antibody selected from the group consisting of: monoclonal antibodies, polyclonal antibodies, humanized antibodies, recombinant antibodies, chemically modified antibodies, and synthetic antibodies.

16. The method of claim 14 wherein α(PTPζ) is an antibody fragment selected from the group consisting of fragments of: monoclonal antibodies, polyclonal antibodies, humanized antibodies, recombinant antibodies, chemically modified antibodies, and synthetic antibodies.

17. The method of claim 12 wherein α(PTPζ) comprises a cytotoxic moiety.

18. The method of claim 17 wherein the cytotoxic moiety comprises a pharmaceutically acceptable radioactive isotope.

19. The method of claim 17 wherein the cytotoxic moiety is chemotoxic.

20. The method of claim 17 wherein the cytotoxic moiety is a toxin protein.

21. The method of claim 12, wherein said antibody has a binding affinity of at least 10 nM.

22. The method of claim 12, wherein said antibody binds to the epitope recognized by one of 1B9G4; 7A9B5; or 7E4B11 monoclonal antibodies.

23. A method for visualizing a brain tumor in a patient, the method comprising:
a) administering to a patient an effective amount of an imaging composition comprising: a compound of the general formula α(PTPζ)I, wherein α(PTPζ) specifically binds the extracellular domain of human protein tyrosine phosphatase-zeta, and I increases contrast between a tumor and surround tissue in a visualization method, and a pharmaceutically acceptable carrier; and
b) visualizing said imaging composition.

24. The method of claim 23 wherein the brain tumor is a glioblastoma.

25. The method of claim 23 wherein I is a radiographic moiety.

26. A purified antibody produced by hybridoma cell line 1B9G4; 7A9B5; or 7E4B11.


SEQUENCE LISTING (14 sequences)

SEQ ID NO: 1 - DNA, 7941 bp, Homo Sapiens
  CDS (148)...(7092)
cacacatacgcacgcacgatctcacttcgatctatacactggaggattaaaacaaacaaacaaaaaaaacatttccttcgctccccctccctctccactctgagaagcagaggagccgcacggcgaggggccgcagaccgtctggaaatgcgaatcctaaagcgtttcctcgcttgcattcagctcctctgtgtttgccgcctggattgggctaatggatactacagacaacagagaaaacttgttgaagagattggctggtcctatacaggagcactgaatcaaaaaaattggggaaagaaatatccaacatgtaatagcccaaaacaatctcctatcaatattgatgaagatcttacacaagtaaatgtgaatcttaagaaacttaaatttcagggttgggataaaacatcattggaaaacacattcattcataacactgggaaaacagtggaaattaatctcactaatgactaccgtgtcagcggaggagtttcagaaatggtgtttaaagcaagcaagataacttttcactggggaaaatgcaatatgtcatctgatggatcagagcatagtttagaaggacaaaaatttccacttgagatgcaaatctactgctttgatgcggaccgattttcaagttttgaggaagcagtcaaaggaaaagggaagttaagagctttatccattttgtttgaggttgggacagaagaaaatttggatttcaaagcgattattgatggagtcgaaagtgttagtcgttttgggaagcaggctgctttagatccattcatactgttgaaccttctgccaaactcaactgacaagtattacatttacaatggctcattgacatctcctccctgcacagacacagttgactggattgtttttaaagatacagttagcatctctgaaagccagttggctgttttttgtgaagttcttacaatgcaacaatctggttatgtcatgctgatggactacttacaaaacaattttcgagagcaacagtacaagttctctagacaggtgttttcctcatacactggaaaggaagagattcatgaagcagtttgtagttcagaaccagaaaatgttcaggctgacccagagaattataccagccttcttgttacatgggaaagacctcgagtcgtttatgataccatgattgagaagtttgcagttttgtaccagcagttggatggagaggaccaaaccaagcatgaatttttgacagatggctatcaagacttgggtgctattctcaataatttgctacccaatatgagttatgttcttcagatagtagccatatgcactaatggcttatatggaaaatacagcgaccaactgattgtcgacatgcctactgataatcctgaacttgatcttttccctgaattaattggaactgaagaaataatcaaggaggaggaagagggaaaagacattgaagaaggcgctattgtgaatcctggtagagacagtgctacaaaccaaatcaggaaaaaggaaccccagatttctaccacaacacactacaatcgcatagggacgaaatacaatgaagccaagactaaccgatccccaacaagaggaagtgaattctctggaaagggtgatgttcccaatacatctttaaattccacttcccaaccagtcactaaattagccacagaaaaagatatttccttgacttctcagactgtgactgaactgccacctcacactgtggaaggtacttcagcctctttaaatgatggctctaaaactgttcttagatctccacatatgaacttgtcggggactgcagaatccttaaatacagtttctataacagaatatgaggaggagagtttattgaccagtttcaagcttgatactggagctgaagattcttcaggctccagtcccgcaacttctgctatcccattcatctctgagaacatatcccaagggtatatattttcctccgaaaacccagagacaataacatatgatgtccttataccagaatctgctagaaatgcttccgaagattcaacttcatcaggttcagaagaatcactaaaggatccttctatggagggaaatgtgtggtttcctagctctacagacataacagcacagcccgatgttggatcaggcagagagagctttctccagactaattacactgagatacgtgttgatgaatctgagaagacaaccaagtccttttctgcaggcccagtgatgtcacagggtccctcagttacagatctggaaatgccacattattctacctttgcctacttcccaactgaggtaacacctcatgcttttaccccatcctccagacaacaggatttggtctccacggtcaacgtggtatactcgcagacaacccaaccggtatacaatggtgagacacctcttcaaccttcctacagtagtgaagtctttcctctagtcacccctttgttgcttgacaatcagatcctcaacactacccctgctgcttcaagtagtgattcggccttgcatgctacgcctgtatttcccagtgtcgatgtgtcatttgaatccatcctgtcttcctatgatggtgcacctttgcttccattttcctctgcttccttcagtagtgaattgtttcgccatctgcatacagtttctcaaatccttccacaagttacttcagctaccgagagtgataaggtgcccttgcatgcttctctgccagtggctgggggtgatttgctattagagcccagccttgctcagtattctgatgtgctgtccactactcatgctgcttcagagacgctggaatttggtagtgaatctggtgttctttataaaacgcttatgttttctcaagttgaaccacccagcagtgatgccatgatgcatgcacgttcttcagggcctgaaccttcttatgccttgtctgataatgagggctcccaacacatcttcactgtttcttacagttctgcaatacctgtgcatgattctgtgggtgtaacttatcagggttccttatttagcggccctagccatataccaatacctaagtcttcgttaataaccccaactgcatcattactgcagcctactcatgccctctctggtgatggggaatggtctggagcctcttctgatagtgaatttcttttacctgacacagatgggctgacagcccttaacatttcttcacctgtttctgtagctgaatttacatatacaacatctgtgtttggtgatgataataaggcgctttctaaaagtgaaataatatatggaaatgagactgaactgcaaattccttctttcaatgagatggtttacccttctgaaagcacagtcatgcccaacatgtatgataatgtaaataagttgaatgcgtctttacaagaaacctctgtttccatttctagcaccaagggcatgtttccagggtcccttgctcataccaccactaaggtttttgatcatgagattagtcaagttccagaaaataacttttcagttcaacctacacatactgtctctcaagcatctggtgacacttcgcttaaacctgtgcttagtgcaaactcagagccagcatcctctgaccctgcttctagtgaaatgttatctccttcaactcagctcttattttatgagacctcagcttcttttagtactgaagtattgctacaaccttcctttcaggcttctgatgttgacaccttgcttaaaactgttcttccagctgtgcccagtgatccaatattggttgaaacccccaaagttgataaaattagttctacaatgttgcatctcattgtatcaaattctgcttcaagtgaaaacatgctgcactctacatctgtaccagtttttgatgtgtcgcctacttctcatatgcactctgcttcacttcaaggtttgaccatttcctatgcaagtgagaaatatgaaccagttttgttaaaaagtgaaagttcccaccaagtggtaccttctttgtacagtaatgatgagttgttccaaacggccaatttggagattaaccaggcccatcccccaaaaggaaggcatgtatttgctacacctgttttatcaattgatgaaccattaaatacactaataaataagcttatacattccgatgaaattttaacctccaccaaaagttctgttactggtaaggtatttgctggtattccaacagttgcttctgatacatttgtatctactgatcattctgttcctataggaaatgggcatgttgccattacagctgtttctccccacagagatggttctgtaacctcaacaaagttgctgtttccttctaaggcaacttctgagctgagtcatagtgccaaatctgatgccggtttagtgggtggtggtgaagatggtgacactgatgatgatggtgatgatgatgatgacagagatagtgatggcttatccattcataagtgtatgtcatgctcatcctatagagaatcacaggaaaaggtaatgaatgattcagacacccacgaaaacagtcttatggatcagaataatccaatctcatactcactatctgagaattctgaagaagataatagagtcacaagtgtatcctcagacagtcaaactggtatggacagaagtcctggtaaatcaccatcagcaaatgggctatcccaaaagcacaatgatggaaaagaggaaaatgacattcagactggtagtgctctgcttcctctcagccctgaatctaaagcatgggcagttctgacaagtgatgaagaaagtggatcagggcaaggtacctcagatagccttaatgagaatgagacttccacagatttcagttttgcagacactaatgaaaaagatgctgatgggatcctggcagcaggtgactcagaaataactcctggattcccacagtccccaacatcatctgttactagcgagaactcagaagtgttccacgtttcagaggcagaggccagtaatagtagccatgagtctcgtattggtctagctgaggggttggaatccgagaagaaggcagttataccccttgtgatcgtgtcagccctgacttttatctgtctagtggttcttgtgggtattctcatctactggaggaaatgcttccagactgcacacttttacttagaggacagtacatcccctagagttatatccacacctccaacacctatctttccaatttcagatgatgtcggagcaattccaataaagcactttccaaagcatgttgcagatttacatgcaagtagtgggtttactgaagaatttgagacactgaaagagttttaccaggaagtgcagagctgtactgttgacttaggtattacagcagacagctccaaccacccagacaacaagcacaagaatcgatacataaatatcgttgcctatgatcatagcagggttaagctagcacagcttgctgaaaaggatggcaaactgactgattatatcaatgccaattatgttgatggctacaacagaccaaaagcttatattgctgcccaaggcccactgaaatccacagctgaagatttctggagaatgatatgggaacataatgtggaagttattgtcatgataacaaacctcgtggagaaaggaaggagaaaatgtgatcagtactggcctgccgatgggagtgaggagtacgggaactttctggtcactcagaagagtgtgcaagtgcttgcctattatactgtgaggaattttactctaagaaacacaaaaataaaaaagggctcccagaaaggaagacccagtggacgtgtggtcacacagtatcactacacgcagtggcctgacatgggagtaccagagtactccctgccagtgctgacctttgtgagaaaggcagcctatgccaagcgccatgcagtggggcctgttgtcgtccactgcagtgctggagttggaagaacaggcacatatattgtgctagacagtatgttgcagcagattcaacacgaaggaactgtcaacatatttggcttcttaaaacacatccgttcacaaagaaattatttggtacaaactgaggagcaatatgtcttcattcatgatacactggttgaggccatacttagtaaagaaactgaggtgctggacagtcatattcatgcctatgttaatgcactcctcattcctggaccagcaggcaaaacaaagctagagaaacaattccagctcctgagccagtcaaatatacagcagagtgactattctgcagccctaaagcaatgcaacagggaaaagaatcgaacttcttctatcatccctgtggaaagatcaagggttggcatttcatccctgagtggagaaggcacagactacatcaatgcctcctatatcatgggctattaccagagcaatgaattcatcattacccagcaccctctccttcataccatcaaggatttctggaggatgatatgggaccataatgcccaactggtggttatgattcctgatggccaaaacatggcagaagatgaatttgtttactggccaaataaagatgagcctataaattgtgagagctttaaggtcactcttatggctgaagaacacaaatgtctatctaatgaggaaaaacttataattcaggactttatcttagaagctacacaggatgattatgtacttgaagtgaggcactttcagtgtcctaaatggccaaatccagatagccccattagtaaaacttttgaacttataagtgttataaaagaagaagctgccaatagggatgggcctatgattgttcatgatgagcatggaggagtgacggcaggaactttctgtgctctgacaacccttatgcaccaactagaaaaagaaaattccgtggatgtttaccaggtagccaagatgatcaatctgatgaggccaggagtctttgctgacattgagcagtatcagtttctctacaaagtgatcctcagccttgtgagcacaaggcaggaagagaatccatccacctctctggacagtaatggtgcagcattgcctgatggaaatatagctgagagcttagagtctttagtttaacacagaaaggggtggggggactcacatctgagcattgttttcctcttcctaaaattaggcaggaaaatcagtctagttctgttatctgttgatttcccatcacctgacagtaactttcatgacataggattctgccgccaaatttatatcattaacaatgtgtgcctttttgcaagacttgtaatttacttattatgtttgaactaaaatgattgaattttacagtatttctaagaatggaattgtggtatttttttctgtattgattttaacagaaaatttcaatttatagaggttaggaattccaaactacagaaaatgtttgtttttagtgtcaaatttttagctgtatttgtagcaattatcaggtttgctagaaatataacttttaatacagtagcctgtaaataaaacactcttccatatgatattcaacattttacaactgcagtattcacctaaagtagaaataatctgttacttattgtaaatactgccctagtgtctccatggaccaaatttatatttataattgtagatttttatattttactactgagtcaagttttctagttctgtgtaattgtttagtttaatgacgtagttcattagctggtcttactctaccagttttctgacattgtattgtgttacctaagtcattaactttgtttcagcatgtaattttaacttttgtggaaaatagaaataccttcattttgaaagaagtttttatgagaataacaccttaccaaacattgttcaaatggtttttatccaaggaattgcaaaaataaatataaatattgccattaaaaaaaaaaaaaaaaaaaaaaaaaaaaaaaaaaa

SEQ ID NO: 2 - protein, 2314 aa, Homo Sapiens
MRILKRFLACIQLLCVCRLDWANGYYRQQRKLVEEIGWSYTGALNQKNWGKKYPTCNSPKQSPINIDEDLTQVNVNLKKLKFQGWDKTSLENTFIHNTGKTVEINLTNDYRVSGGVSEMVFKASKITFHWGKCNMSSDGSEHSLEGQKFPLEMQIYCFDADRFSSFEEAVKGKGKLRALSILFEVGTEENLDFKAIIDGVESVSRFGKQAALDPFILLNLLPNSTDKYYIYNGSLTSPPCTDTVDWIVFKDTVSISESQLAVFCEVLTMQQSGYVMLMDYLQNNFREQQYKFSRQVFSSYTGKEEIHEAVCSSEPENVQADPENYTSLLVTWERPRVVYDTMIEKFAVLYQQLDGEDQTKHEFLTDGYQDLGAILNNLLPNMSYVLQIVAICTNGLYGKYSDQLIVDMPTDNPELDLFPELIGTEEIIKEEEEGKDIEEGAIVNPGRDSATNQIRKKEPQISTTTHYNRIGTKYNEAKTNRSPTRGSEFSGKGDVPNTSLNSTSQPVTKLATEKDISLTSQTVTELPPHTVEGTSASLNDGSKTVLRSPHMNLSGTAESLNTVSITEYEEESLLTSFKLDTGAEDSSGSSPATSAIPFISENISQGYIFSSENPETITYDVLIPESARNASEDSTSSGSEESLKDPSMEGNVWFPSSTDITAQPDVGSGRESFLQTNYTEIRVDESEKTTKSFSAGPVMSQGPSVTDLEMPHYSTFAYFPTEVTPHAFTPSSRQQDLVSTVNVVYSQTTQPVYNGETPLQPSYSSEVFPLVTPLLLDNQILNTTPAASSSDSALHATPVFPSVDVSFESILSSYDGAPLLPFSSASFSSELFRHLHTVSQILPQVTSATESDKVPLHASLPVAGGDLLLEPSLAQYSDVLSTTHAASETLEFGSESGVLYKTLMFSQVEPPSSDAMMHARSSGPEPSYALSDNEGSQHIFTVSYSSAIPVHDSVGVTYQGSLFSGPSHIPIPKSSLITPTASLLQPTHALSGDGEWSGASSDSEFLLPDTDGLTALNISSPVSVAEFTYTTSVFGDDNKALSKSEIIYGNETELQIPSFNEMVYPSESTVMPNMYDNVNKLNASLQETSVSISSTKGMFPGSLAHTTTKVFDHEISQVPENNFSVQPTHTVSQASGDTSLKPVLSANSEPASSDPASSEMLSPSTQLLFYETSASFSTEVLLQPSFQASDVDTLLKTVLPAVPSDPILVETPKVDKISSTMLHLIVSNSASSENMLHSTSVPVFDVSPTSHMHSASLQGLTISYASEKYEPVLLKSESSHQVVPSLYSNDELFQTANLEINQAHPPKGRHVFATPVLSIDEPLNTLINKLIHSDEILTSTKSSVTGKVFAGIPTVASDTFVSTDHSVPIGNGHVAITAVSPHRDGSVTSTKLLFPSKATSELSHSAKSDAGLVGGGEDGDTDDDGDDDDDRDSDGLSIHKCMSCSSYRESQEKVMNDSDTHENSLMDQNNPISYSLSENSEEDNRVTSVSSDSQTGMDRSPGKSPSANGLSQKHNDGKEENDIQTGSALLPLSPESKAWAVLTSDEESGSGQGTSDSLNENETSTDFSFADTNEKDADGILAAGDSEITPGFPQSPTSSVTSENSEVFHVSEAEASNSSHESRIGLAEGLESEKKAVIPLVIVSALTFICLVVLVGILIYWRKCFQTAHFYLEDSTSPRVISTPPTPIFPISDDVGAIPIKHFPKHVADLHASSGFTEEFETLKEFYQEVQSCTVDLGITADSSNHPDNKHKNRYINIVAYDHSRVKLAQLAEKDGKLTDYINANYVDGYNRPKAYIAAQGPLKSTAEDFWRMIWEHNVEVIVMITNLVEKGRRKCDQYWPADGSEEYGNFLVTQKSVQVLAYYTVRNFTLRNTKIKKGSQKGRPSGRVVTQYHYTQWPDMGVPEYSLPVLTFVRKAAYAKRHAVGPVVVHCSAGVGRTGTYIVLDSMLQQIQHEGTVNIFGFLKHIRSQRNYLVQTEEQYVFIHDTLVEAILSKETEVLDSHIHAYVNALLIPGPAGKTKLEKQFQLLSQSNIQQSDYSAALKQCNREKNRTSSIIPVERSRVGISSLSGEGTDYINASYIMGYYQSNEFIITQHPLLHTIKDFWRMIWDHNAQLVVMIPDGQNMAEDEFVYWPNKDEPINCESFKVTLMAEEHKCLSNEEKLIIQDFILEATQDDYVLEVRHFQCPKWPNPDSPISKTFELISVIKEEAANRDGPMIVHDEHGGVTAGTFCALTTLMHQLEKENSVDVYQVAKMINLMRPGVFADIEQYQFLYKVILSLVSTRQEENPSTSLDSNGAALPDGNIAESLESLV

SEQ ID NO: 3 - DNA, 3091 bp, Homo sapiens
  CDS (148)...(1272): PTP-zeta SM1 exon 9 variant
cacacatacgcacgcacgatctcacttcgatctatacactggaggattaaaacaaacaaacaaaaaaaacatttccttcgctccccctccctctccactctgagaagcagaggagccgcacggcgaggggccgcagaccgtctggaaatgcgaatcctaaagcgtttcctcgcttgcattcagctcctctgtgtttgccgcctggattgggctaatggatactacagacaacagagaaaacttgttgaagagattggctggtcctatacaggagcactgaatcaaaaaaattggggaaagaaatatccaacatgtaatagcccaaaacaatctcctatcaatattgatgaagatcttacacaagtaaatgtgaatcttaagaaacttaaatttcagggttgggataaaacatcattggaaaacacattcattcataacactgggaaaacagtggaaattaatctcactaatgactaccgtgtcagcggaggagtttcagaaatggtgtttaaagcaagcaagataacttttcactggggaaaatgcaatatgtcatctgatggatcagagcatagtttagaaggacaaaaatttccacttgagatgcaaatctactgctttgatgcggaccgattttcaagttttgaggaagcagtcaaaggaaaagggaagttaagagctttatccattttgtttgaggttgggacagaagaaaatttggatttcaaagcgattattgatggagtcgaaagtgttagtcgttttgggaagcaggctgctttagatccattcatactgttgaaccttctgccaaactcaactgacaagtattacatttacaatggctcattgacatctcctccctgcacagacacagttgactggattgtttttaaagatacagttagcatctctgaaagccagttggctgttttttgtgaagttcttacaatgcaacaatctggttatgtcatgctgatggactacttacaaaacaattttcgagagcaacagtacaagttctctagacaggtgttttcctcatacactggaaaggaagagattcatgaagcagtttgtagttcagaaccagaaaatgttcaggctgacccagagaattataccagccttcttgttacatgggaaagacctcgagtcgtttatgataccatgattgagaagtttgcagttttgtaccagcagttggatggagaggaccaaaccaagcatgaatttttgacagatggctatcaagacttggtaactatatgatcagttgttttacatagggtaacattataatttaatttccaaggtaagaacttacaaatggttgtatattattttcctccattacttttagactttatgtgaaggtggggtaggctgagtatttttaaatttaaaaaaaaattttaaattagaagctatactaaattatgtttaaagttacatttaattaaatggatatcataactttgccaacaataacactatagagtagatacatatgacttatgaactggagatcatttagtgtggcctttcttaagatttcagttgtagaatagtgccagaatctcagtgccctgatacattttatattgtgtcttccattacgctatatcagcacaggaaaagtagagtaggggacatacaagtcctctttgttgcaccaaaaaattttcagataacagctgggaagtcatgattgggtcagaactttggggatgtaagaaaacatttcttacaaaaagatccacccctgcctccctccaccagcgcatgcgaataaagtacagattccctttgtggcctgagcatgtcagtattaaactttgctctggtagggaagtgttggccatagattagggtgtagttgacaaaccttcatctggatgtaggtccagaaagtccccactgcaggttaaaggacactggactctgcactcaggcacctagagtcctgcaagtcctgggaacctgcatttaaataaaaatgcactattaattatgtttcatatcatgtggacaaaatggataaaattttagtaaccttttaattcagttgcctggaatatggagacacaatgacctgggaaaatcgtgaaataaatagtaataaaaatgtttatttcataattacgtgaagaagataattctattactgttcttgcatatatattgtcaagaaaaagagataacttagttgttcactttttcacattgctccttgtttgcaaatgccccccatttatttgtctaaaatattaatttttagtttgtagtactaatttatgaatttgatgagttctggctaaaaatgaaacttcctgaaactaaatctgatttttaaaaagcaaaaaaaaaaaaaaagcctagctttccagttcttcataattcacaaataccacaagtttaactaagcaacattgcataaacttttccttaggttaataaaatagaagtattttccacggaccagggagaaaaagttttctaggaaagatacctagtgtgttggtagtcctatgagaataacatttgtataattactaacatctttcttttagggtgctattctcaataatttgctacccaatatgagttatgttcttcagatagtagccatatgcactaatggcttatatggaaaatacagcgaccaactgattgtcgacatgcctactgataatcctggtaagtgccaccagatacatctatatattaactcaataaatgaggttagtttaattactgtatgcattgatgctttctctctatattcttttggccaaaaggcaaagtgattttctcttaagtctggattgccgggtaattttttggggcatgggacccatttctcattcagcaggtctggtgccagacaataagtaaacttatccttaatattggagtttaccatttgtaaaataagagtgactaaacatatttataacattgtaataatcattaaatgaaaattgctatgtaaatgttgagactgttattttggataattaagagttggtttaatttgtatttatttcctcttttcagcccccaaagcattatgtagtaagtgtataca

SEQ ID NO: 4 - protein, 374 aa, Homo sapiens
MRILKRFLACIQLLCVCRLDWANGYYRQQRKLVEEIGWSYTGALNQKNWGKKYPTCNSPKQSPINIDEDLTQVNVNLKKLKFQGWDKTSLENTFIHNTGKTVEINLTNDYRVSGGVSEMVFKASKITFHWGKCNMSSDGSEHSLEGQKFPLEMQIYCFDADRFSSFEEAVKGKGKLRALSILFEVGTEENLDFKAIIDGVESVSRFGKQAALDPFILLNLLPNSTDKYYIYNGSLTSPPCTDTVDWIVFKDTVSISESQLAVFCEVLTMQQSGYVMLMDYLQNNFREQQYKFSRQVFSSYTGKEEIHEAVCSSEPENVQADPENYTSLLVTWERPRVVYDTMIEKFAVLYQQLDGEDQTKHEFLTDGYQDLVTI

SEQ ID NO: 5 - DNA, 8058 bp, Homo sapiens
  misc_feature (6229)...(6345): Exon 23a splice variant of PTP-zeta SM2
cacacatacgcacgcacgatctcacttcgatctatacactggaggattaaaacaaacaaacaaaaaaaacatttccttcgctccccctccctctccactctgagaagcagaggagccgcacggcgaggggccgcagaccgtctggaaatgcgaatcctaaagcgtttcctcgcttgcattcagctcctctgtgtttgccgcctggattgggctaatggatactacagacaacagagaaaacttgttgaagagattggctggtcctatacaggagcactgaatcaaaaaaattggggaaagaaatatccaacatgtaatagcccaaaacaatctcctatcaatattgatgaagatcttacacaagtaaatgtgaatcttaagaaacttaaatttcagggttgggataaaacatcattggaaaacacattcattcataacactgggaaaacagtggaaattaatctcactaatgactaccgtgtcagcggaggagtttcagaaatggtgtttaaagcaagcaagataacttttcactggggaaaatgcaatatgtcatctgatggatcagagcatagtttagaaggacaaaaatttccacttgagatgcaaatctactgctttgatgcggaccgattttcaagttttgaggaagcagtcaaaggaaaagggaagttaagagctttatccattttgtttgaggttgggacagaagaaaatttggatttcaaagcgattattgatggagtcgaaagtgttagtcgttttgggaagcaggctgctttagatccattcatactgttgaaccttctgccaaactcaactgacaagtattacatttacaatggctcattgacatctcctccctgcacagacacagttgactggattgtttttaaagatacagttagcatctctgaaagccagttggctgttttttgtgaagttcttacaatgcaacaatctggttatgtcatgctgatggactacttacaaaacaattttcgagagcaacagtacaagttctctagacaggtgttttcctcatacactggaaaggaagagattcatgaagcagtttgtagttcagaaccagaaaatgttcaggctgacccagagaattataccagccttcttgttacatgggaaagacctcgagtcgtttatgataccatgattgagaagtttgcagttttgtaccagcagttggatggagaggaccaaaccaagcatgaatttttgacagatggctatcaagacttgggtgctattctcaataatttgctacccaatatgagttatgttcttcagatagtagccatatgcactaatggcttatatggaaaatacagcgaccaactgattgtcgacatgcctactgataatcctgaacttgatcttttccctgaattaattggaactgaagaaataatcaaggaggaggaagagggaaaagacattgaagaaggcgctattgtgaatcctggtagagacagtgctacaaaccaaatcaggaaaaaggaaccccagatttctaccacaacacactacaatcgcatagggacgaaatacaatgaagccaagactaaccgatccccaacaagaggaagtgaattctctggaaagggtgatgttcccaatacatctttaaattccacttcccaaccagtcactaaattagccacagaaaaagatatttccttgacttctcagactgtgactgaactgccacctcacactgtggaaggtacttcagcctctttaaatgatggctctaaaactgttcttagatctccacatatgaacttgtcggggactgcagaatccttaaatacagtttctataacagaatatgaggaggagagtttattgaccagtttcaagcttgatactggagctgaagattcttcaggctccagtcccgcaacttctgctatcccattcatctctgagaacatatcccaagggtatatattttcctccgaaaacccagagacaataacatatgatgtccttataccagaatctgctagaaatgcttccgaagattcaacttcatcaggttcagaagaatcactaaaggatccttctatggagggaaatgtgtggtttcctagctctacagacataacagcacagcccgatgttggatcaggcagagagagctttctccagactaattacactgagatacgtgttgatgaatctgagaagacaaccaagtccttttctgcaggcccagtgatgtcacagggtccctcagttacagatctggaaatgccacattattctacctttgcctacttcccaactgaggtaacacctcatgcttttaccccatcctccagacaacaggatttggtctccacggtcaacgtggtatactcgcagacaacccaaccggtatacaatggtgagacacctcttcaaccttcctacagtagtgaagtctttcctctagtcacccctttgttgcttgacaatcagatcctcaacactacccctgctgcttcaagtagtgattcggccttgcatgctacgcctgtatttcccagtgtcgatgtgtcatttgaatccatcctgtcttcctatgatggtgcacctttgcttccattttcctctgcttccttcagtagtgaattgtttcgccatctgcatacagtttctcaaatccttccacaagttacttcagctaccgagagtgataaggtgcccttgcatgcttctctgccagtggctgggggtgatttgctattagagcccagccttgctcagtattctgatgtgctgtccactactcatgctgcttcagagacgctggaatttggtagtgaatctggtgttctttataaaacgcttatgttttctcaagttgaaccacccagcagtgatgccatgatgcatgcacgttcttcagggcctgaaccttcttatgccttgtctgataatgagggctcccaacacatcttcactgtttcttacagttctgcaatacctgtgcatgattctgtgggtgtaacttatcagggttccttatttagcggccctagccatataccaatacctaagtcttcgttaataaccccaactgcatcattactgcagcctactcatgccctctctggtgatggggaatggtctggagcctcttctgatagtgaatttcttttacctgacacagatgggctgacagcccttaacatttcttcacctgtttctgtagctgaatttacatatacaacatctgtgtttggtgatgataataaggcgctttctaaaagtgaaataatatatggaaatgagactgaactgcaaattccttctttcaatgagatggtttacccttctgaaagcacagtcatgcccaacatgtatgataatgtaaataagttgaatgcgtctttacaagaaacctctgtttccatttctagcaccaagggcatgtttccagggtcccttgctcataccaccactaaggtttttgatcatgagattagtcaagttccagaaaataacttttcagttcaacctacacatactgtctctcaagcatctggtgacacttcgcttaaacctgtgcttagtgcaaactcagagccagcatcctctgaccctgcttctagtgaaatgttatctccttcaactcagctcttattttatgagacctcagcttcttttagtactgaagtattgctacaaccttcctttcaggcttctgatgttgacaccttgcttaaaactgttcttccagctgtgcccagtgatccaatattggttgaaacccccaaagttgataaaattagttctacaatgttgcatctcattgtatcaaattctgcttcaagtgaaaacatgctgcactctacatctgtaccagtttttgatgtgtcgcctacttctcatatgcactctgcttcacttcaaggtttgaccatttcctatgcaagtgagaaatatgaaccagttttgttaaaaagtgaaagttcccaccaagtggtaccttctttgtacagtaatgatgagttgttccaaacggccaatttggagattaaccaggcccatcccccaaaaggaaggcatgtatttgctacacctgttttatcaattgatgaaccattaaatacactaataaataagcttatacattccgatgaaattttaacctccaccaaaagttctgttactggtaaggtatttgctggtattccaacagttgcttctgatacatttgtatctactgatcattctgttcctataggaaatgggcatgttgccattacagctgtttctccccacagagatggttctgtaacctcaacaaagttgctgtttccttctaaggcaacttctgagctgagtcatagtgccaaatctgatgccggtttagtgggtggtggtgaagatggtgacactgatgatgatggtgatgatgatgatgacagagatagtgatggcttatccattcataagtgtatgtcatgctcatcctatagagaatcacaggaaaaggtaatgaatgattcagacacccacgaaaacagtcttatggatcagaataatccaatctcatactcactatctgagaattctgaagaagataatagagtcacaagtgtatcctcagacagtcaaactggtatggacagaagtcctggtaaatcaccatcagcaaatgggctatcccaaaagcacaatgatggaaaagaggaaaatgacattcagactggtagtgctctgcttcctctcagccctgaatctaaagcatgggcagttctgacaagtgatgaagaaagtggatcagggcaaggtacctcagatagccttaatgagaatgagacttccacagatttcagttttgcagacactaatgaaaaagatgctgatgggatcctggcagcaggtgactcagaaataactcctggattcccacagtccccaacatcatctgttactagcgagaactcagaagtgttccacgtttcagaggcagaggccagtaatagtagccatgagtctcgtattggtctagctgaggggttggaatccgagaagaaggcagttataccccttgtgatcgtgtcagccctgacttttatctgtctagtggttcttgtgggtattctcatctactggaggaaatgcttccagactgcacacttttacttagaggacagtacatcccctagagttatatccacacctccaacacctatctttccaatttcagatgatgtcggagcaattccaataaagcactttccaaagcatgttgcagatttacatgcaagtagtgggtttactgaagaatttgagacactgaaagagttttaccaggaagtgcagagctgtactgttgacttaggtattacagcagacagctccaaccacccagacaacaagcacaagaatcgatacataaatatcgttgcctatgatcatagcagggttaagctagcacagcttgctgaaaaggatggcaaactgactgattatatcaatgccaattatgttgatggctacaacagaccaaaagcttatattgctgcccaaggcccactgaaatccacagctgaagatttctggagaatgatatgggaacataatgtggaagttattgtcatgataacaaacctcgtggagaaaggaaggagaaaatgtgatcagtactggcctgccgatgggagtgaggagtacgggaactttctggtcactcagaagagtgtgcaagtgcttgcctattatactgtgaggaattttactctaagaaacacaaaaataaaaaagggctcccagaaaggaagacccagtggacgtgtggtcacacagtatcactacacgcagtggcctgacatgggagtaccagagtactccctgccagtgctgacctttgtgagaaaggcagcctatgccaagcgccatgcagtggggcctgttgtcgtccactgcagtgctggagttggaagaacaggcacatatattgtgctagacagtatgttgcagcagattcaacacgaaggaactgtcaacatatttggcttcttaaaacacatccgttcacaaagaaattatttggtacaaactgaggagcaatatgtcttcattcatgatacactggttgaggccatacttagtaaagaaactgaggtgctggacagtcatattcatgcctatgttaatgcactcctcattcctggaccagcaggcaaaacaaagctagagaaacaattccagggtctcactctgtcacccaggctggagtgcagaggcacaatctcggctcactgcaaccttcctctccctggcttaactgatcctcctacctcagcctcccgagtggctgggactatactcctgagccagtcaaatatacagcagagtgactattctgcagccctaaagcaatgcaacagggaaaagaatcgaacttcttctatcatccctgtggaaagatcaagggttggcatttcatccctgagtggagaaggcacagactacatcaatgcctcctatatcatgggctattaccagagcaatgaattcatcattacccagcaccctctccttcataccatcaaggatttctggaggatgatatgggaccataatgcccaactggtggttatgattcctgatggccaaaacatggcagaagatgaatttgtttactggccaaataaagatgagcctataaattgtgagagctttaaggtcactcttatggctgaagaacacaaatgtctatctaatgaggaaaaacttataattcaggactttatcttagaagctacacaggatgattatgtacttgaagtgaggcactttcagtgtcctaaatggccaaatccagatagccccattagtaaaacttttgaacttataagtgttataaaagaagaagctgccaatagggatgggcctatgattgttcatgatgagcatggaggagtgacggcaggaactttctgtgctctgacaacccttatgcaccaactagaaaaagaaaattccgtggatgtttaccaggtagccaagatgatcaatctgatgaggccaggagtctttgctgacattgagcagtatcagtttctctacaaagtgatcctcagccttgtgagcacaaggcaggaagagaatccatccacctctctggacagtaatggtgcagcattgcctgatggaaatatagctgagagcttagagtctttagtttaacacagaaaggggtggggggactcacatctgagcattgttttcctcttcctaaaattaggcaggaaaatcagtctagttctgttatctgttgatttcccatcacctgacagtaactttcatgacataggattctgccgccaaatttatatcattaacaatgtgtgcctttttgcaagacttgtaatttacttattatgtttgaactaaaatgattgaattttacagtatttctaagaatggaattgtggtatttttttctgtattgattttaacagaaaatttcaatttatagaggttaggaattccaaactacagaaaatgtttgtttttagtgtcaaatttttagctgtatttgtagcaattatcaggtttgctagaaatataacttttaatacagtagcctgtaaataaaacactcttccatatgatattcaacattttacaactgcagtattcacctaaagtagaaataatctgttacttattgtaaatactgccctagtgtctccatggaccaaatttatatttataattgtagatttttatattttactactgagtcaagttttctagttctgtgtaattgtttagtttaatgacgtagttcattagctggtcttactctaccagttttctgacattgtattgtgttacctaagtcattaactttgtttcagcatgtaattttaacttttgtggaaaatagaaataccttcattttgaaagaagtttttatgagaataacaccttaccaaacattgttcaaatggtttttatccaaggaattgcaaaaataaatataaatattgccattaaaaaaaaaaaaaaaaaaaaaaaaaaaaaaaaaaa

SEQ ID NO: 6 - protein, 2353 aa, Homo sapiens [sequence omitted]

SEQ ID NO: 7 - DNA, 22 bp, Homo sapiens
cagcagttggatggaagaggac

SEQ ID NO: 8 - DNA, 22 bp, Homo sapiens
cactgagattctggcactattc

SEQ ID NO: 9 - DNA, 21 bp, Homo sapiens
aacaattccagggtctcactc

SEQ ID NO: 10 - DNA, 21 bp, Homo sapiens
ttgactggctcaggagtatag

SEQ ID NO: 11 - DNA, 21 bp, Homo sapiens
ctgataatgagggctcccaac

SEQ ID NO: 12 - DNA, 24 bp, Homo sapiens
ctctgcacttcctggtaaaactct

SEQ ID NO: 13 - DNA, 22 bp, Homo sapiens
cagcagttggatggaagaggac

SEQ ID NO: 14 - DNA, 24 bp, Homo sapiens
ctctgcacttcctggtaaaactct